(19) United States
(12) Patent Application Publication
(10) Pub. No.: US 2022/0175962 A1
(43) Pub. Date: Jun. 9, 2022

(54) GENE THERAPY COMPOSITIONS AND METHODS FOR TREATING PARKINSON'S DISEASE

(71) Applicants: SIO GENE THERAPIES INC., New York, NY (US); OXFORD BIOMEDICA LTD., Oxford (GB)

(72) Inventors: Gavin CORCORAN, New York, NY (US); Michele STONE, Durham, NC (US); Kyriacos MITROPHANOUS, Oxford (GB); Hannah STEWART, Oxford (GB)

(21) Appl. No.: 17/438,229

(22) PCT Filed: Mar. 10, 2020
(86) PCT No.: PCT/IB2020/052073

§ 371 Date: Sep. 10, 2021

Related U.S. Application Data
(60) Provisional application No. 62/816,170, filed on Mar. 10, 2019.
(60) Provisional application No. 62/871,007, filed on Jul. 5, 2019.

(51) Int. Cl. A61K 48/00; A61P 25/16

(52) U.S. Cl. CPC A61K 48/005; A61P 25/16

(57) ABSTRACT

A method of improving motor function and reducing dyskinesia in a subject suffering from a neurodegenerative disease or a disease where endogenous dopamine levels are reduced in the subject comprising administering an effective amount of a viral vector comprising a nucleic acid construct comprising (i) a nucleotide sequence which encodes tyrosine hydroxylase (TH), (ii) a nucleotide sequence which encodes GTP-cyclohydrolase I (CH1), (iii) a nucleotide sequence which encodes Aromatic Amino Acid Dopa Decarboxylase (AADC), or any combination thereof to the subject.

### CROSS-REFERENCE TO RELATED APPLICATIONS

This application claims the benefit of U.S. Provisional Patent Application No. 62/816,170, filed Mar. 10, 2019; and U.S. Provisional Patent Application No. 62/871,007, filed Jul. 5, 2019, each of which is incorporated herein by reference in its entirety.

### REFERENCE TO SEQUENCE LISTING SUBMITTED ELECTRONICALLY VIA EFS-WEB

The content of the electronically submitted sequence listing (Name: 4226_038PC02_seqListing_ST25.txt, Size: 4,044 bytes; and Date of Creation: Mar. 10, 2020) submitted with this application is incorporated herein by reference in its entirety.

### FIELD

The present disclosure relates to gene therapy compositions comprising nucleotide sequences encoding enzyme activities involved in the dopamine synthesis pathway delivered using a viral vector particle for treatment of Parkinson's Disease.

### BACKGROUND

Parkinson's disease (PD) is a neurodegenerative disorder of the central nervous system (CNS) that is characterized by the loss of dopaminergic neurons in the substantia nigra. This ultimately leads to dopamine depletion in the striatum causing severe motor deficits. The cause of PD for most patients is unknown, and this is referred to as ‘sporadic’ or ‘idiopathic’ PD. It is estimated that 6.3 million people worldwide have PD, and although most people will develop the symptoms after 60 years of age, approximately one in ten are diagnosed before the age of 50 (Available at European Parkinson's Disease Association website: www.epda.eu.com/en/pd-info/). Slightly more men than women are affected.

There is no curative treatment or therapy to halt the disease progression of PD. Current treatment options include pharmacological and surgical approaches. The early stages of disease can be effectively managed by oral dopaminergic treatments, such as L-DOPA (the precursor to dopamine) therapy, dopamine agonists and enzyme inhibitors that aim to stop the breakdown of L-DOPA or dopamine in the periphery. After approximately 5 years of oral dopaminergic treatments, 50% of patients develop motor problems, such as dyskinesias, in particular after prolonged and pulsatile use. For example, as the disease progresses, L-DOPA therapy becomes less effective in the treatment of the motor deficits, requiring higher doses to be used which have severe side effects.

Once the oral drugs start to fail in mid to late stage PD there is no standard care. At this stage more invasive surgical therapies to control motor functions and to reduce hypomobility episodes are introduced, including deep brain stimulation (DBS), Duodopa® (combined levodopa/carbidopa) and apomorphine (dopamine agonist) pumps.

### DESCRIPTION OF THE FIGURES

FIG. 1 shows a schematic endogenous dopamine synthesis including enzymes Tyrosine hydroxylase (TH) and Cyclohydrolase 1 (CH1), which converts tyrosine to levodopa (L-dopa); and Aromatic L-amino acid decarboxylase (AADC), which converts L-dopa to dopamine.

FIGS. 2A-2B show the gene constructs for (A) Lenti-PD and (B) ProSavin®, respectively. Differences between the two constructions include that in Lenti-PD, CH1 is moved closer to the promoter to enhance expression, and in Lenti-PD, TH and CH1 are joined by a flexible linker to ensure co-localization.

FIG. 3 shows in vitro production of dopamine & L-dopa in primary human neurons with ProSavin® and Lenti-PD.

FIGS. 4A-4B show the phase 2 clinical study design including (A) Part A: Dose Escalation and (B) Part B: Expansion Cohort v. Imitation Surgical Procedure.

FIG. 5 shows PD patient progression reflected through UPDRS part III (motor) OFF score. Cohort 1 subjects has baselines score of 58 and 60.

FIG. 6 shows the change from baseline in the UPDRS Part III OFF score at month 3 for the low dose cohort of Lenti-PD as well as for the low, middle, and high dose cohorts of ProSavin® and Sham control from a prior clinical trial.

FIGS. 7A-7B shows UPDRS OFF change from baseline across subscales, (A) Activities of Daily Living and (B) Complications of Therapy, at month 3 for the low dose cohort of Lenti-PD as well as for the all cohorts of ProSavin®.

FIG. 8 shows Hauser Patient Diaries and Levodopa Equivalent Dose (LED). Hauser Patient Diaries were collected over 2 days just prior to the study visit. The patient was required to complete the diary every 30 mins for each 24-hour period.

FIGS. 9A-9D. FIG. 9A shows the change from baseline in the UPDRS Part III OFF score at month 3 and month 6 for the low dose cohort of Lenti-PD as well as for the low, middle, and high dose cohorts of ProSavin® and Sham control from a prior clinical trial. FIGS. 9B and 9C show UPDRS OFF change from baseline across subscales, (B) Activities of Daily Living and (C) Complications of Therapy, at month 3 and month 6 for the low dose cohort of Lenti-PD as well as for the all cohorts of ProSavin®. FIG. 9D shows the results of a PDQ-39 Summary index as compared to ProSavin®. PDQ-39 is a questionnaire that assesses Parkinson's disease-specific health related quality. Patients experienced an average improvement of 19.5 points from baseline on the UPDRS II Activities of Daily Living “OFF” score, 3 points from baseline on the UPDRS IV Complications of Therapy “OFF” score, and 32.1 points from baseline on the PDQ-39 summary index score at six months.

FIGS. 10A-10B show the clinical rating scores and quantification of the percent change in the total distance moved (TDM) in the MPTP macaque model of PD post-vector administration. The percentages were calculated from the last 3 TDM values obtained at each time-point. The data are relative to baseline (for post-MPTP) or relative to post-MPTP values (3 and 6 month data). Data represent mean±s.e.m.; p≤0.002***

### SUMMARY OF ASPECTS OF THE INVENTION

Certain aspects of the disclosure are directed to a method of improving motor function and reducing dyskinesia in a subject suffering from a neurodegenerative disease or a disease where endogenous dopamine levels are reduced in the subject comprising administering an effective amount of a viral vector comprising a nucleic acid construct comprising (i) a nucleotide sequence which encodes tyrosine hydroxylase (TH), (ii) a nucleotide sequence which encodes GTP-cyclohydrolase I (CH1), (iii) a nucleotide sequence which encodes Aromatic Amino Acid Dopa Decarboxylase (AADC), or any combination thereof to the subject. In some embodiments, the neurodegenerative disease or the disease where endogenous dopamine levels are reduced is Parkinson's Disease.

In some aspects of the disclosure are directed to a method of treating or improving motor function and reducing dyskinesia in a subject suffering from Parkinson's Disease comprising administering to the subject a therapeutically effective amount of a composition which comprises: (a) a viral vector comprising a nucleic acid construct comprising (i) a nucleotide sequence which encodes tyrosine hydroxylase (TH), (ii) a nucleotide sequence which encodes GTP-cyclohydrolase I (CH1), (iii) a nucleotide sequence which encodes Aromatic Amino Acid Dopa Decarboxylase (AADC), or any combination thereof; and (b) a pharmaceutically acceptable excipient.

In some embodiments, the subject is undergoing L-DOPA or levodopa equivalent dose (LED) therapy. In some embodiments, the subject's daily L-DOPA or LED therapy dose is reduced within three months after administration of the nucleic acid construct relative to the subject's L-DOPA or LED therapy dose prior to administration. In some embodiments, the average daily L-DOPA or LED therapy dose is reduced by at least 10%, at least 12%, at least 14%, at least 15%, at least 16%, at least 17%, at least 18%, at least 19%, or at least 20% from baseline three months after administration.

In some embodiments, the viral vector is administered at a target dose of 1×10⁶ TU/subject to 5×10⁸ TU/subject. In some embodiments, the target dose is about 4×10⁶ TU/subject to 8×10⁶ TU/subject; 8×10⁶ TU/subject to 4×10⁷ TU/subject; or 1×10⁷ TU/subject to 5×10⁸ TU/subject.

In some embodiments, the administration is a one-time administration. In some embodiments, the administration is to the brain. In some embodiments, the administration is to the putamen by infusion.

In some embodiments, the nucleic acid construct comprises: (i) a nucleotide sequence which encodes tyrosine hydroxylase (TH), (ii) a nucleotide sequence which encodes GTP-cyclohydrolase I (CH1) and (iii) a nucleotide sequence which encodes Aromatic Amino Acid Dopa Decarboxylase (AADC) wherein the nucleotide sequence encoding TH is linked to the nucleotide sequence encoding CH1 such that they encode a fusion protein TH-CH1.

In some embodiments, the nucleic acid construct comprises:

TH-_{L}-CH1-_{IRES}-AADC;

AADC-_{L}-TH-_{L}-CH1;

TH-_{L}-CH1-_{L}-AADC;

or TH-_{L}-CH1-_{L}-AADC;

wherein L is a linker-encoding sequence, IRES is an Internal Ribosome Entry Site, and P is a promoter.

In some embodiments, the nucleic acid construct comprises TH-L-CH1-IRES-AADC.

In some embodiments, the nucleic acid construct comprises (i) a nucleotide sequence which encodes tyrosine hydroxylase (TH), (ii) a nucleotide sequence which encodes GTP-cyclohydrolase I (CH1) and (iii) a nucleotide sequence which encodes Aromatic Amino Acid Dopa Decarboxylase (AADC), wherein the nucleotide sequence encoding TH is linked to the nucleotide sequence encoding CH1 such that they encode a fusion protein TH-CH1, wherein the construct comprises TH-L-CH1-IRES-AADC or TH-L-CH1-P-AADC, wherein L is a linker-encoding sequence, IRES is an Internal Ribosome Entry Site, and P is a promoter.

In some embodiments, the linker (L) comprises the sequence shown as SEQ ID NO: 1 or SEQ ID NO: 3. In some embodiments, the construct further comprises a promoter (P) selected from a constitutive promoter, a tissue-specific promoter, or a combination thereof. In some embodiments, the promoter is a CMV promoter, a phosphoglycerate kinase promoter or a thymidine kinase promoter.

In some embodiments, the viral vector is a lentiviral vector or an adeno-associated viral vector. In some embodiments, the viral vector is a lentiviral vector. In some embodiments, the viral vector is a viral particle. In some embodiments, the viral particle is an EIAV vector particle, and which is pseudotyped with VSV-G.

In some embodiments, the viral vector is formulated as pharmaceutical composition comprising a pharmaceutically acceptable excipient and/or diluent.

In some embodiments, the motor function in the subject is improved as shown by at least a 25%, at least a 30%, at least a 35%, at least a 40%, at least 45%, or at least 50% increase in UPDRS-III (motor) OFF score 3 months after administration compared to baseline.

In some embodiments, dyskinesias in the subject is reduced in the subject as shown by one or more of the following: (a) at least 50%, at least 60%, at least 70%, at least 75%, at least 80%, at least 85%, or at least 90% reduction in Hauser diary ON time with troublesome dyskinesia 3 months after administration compared to baseline; (b) at least 50% reduction, at least 60%, at least 65%, at least 70%, at least 74%, or at least 75% reduction in UPDRS-IV score 3 months after administration compared to baseline, and (c) at least 10%, at least 12%, at least 15%, at least 18%, at least 20%, or at least 25% improvement in Rush Dyskinesia score 3 months after administration compared to baseline.

In some embodiments, the method further improves one or more symptoms in the subject selected from the group consisting of tremors, bradykinesia, rigid muscles, impaired posture and/or balance, loss of automatic movements, difficulty speaking, difficulty with fine motor skills, or any combination thereof.

In some embodiments, within three months after administration, the subject has improved motor function, reduced dyskinesia, and reduced L-DOPA or LED therapy dose after administration relative to the subject's baseline before administration.

### DETAILED DESCRIPTION

### Definitions

The following definitions and methods are provided to better define the present invention and to guide those of ordinary skill in the art in the practice of the present invention. It must be noted that as used herein and in the appended claims, the singular forms “a” or “an” or “the” include plural referents unless the context clearly dictates otherwise. Thus, for example, reference to “an enzyme” includes a plurality of enzymes.

As used herein, in some embodiments, the term “vector” is used in reference to nucleic acid molecules that transfer nucleic acid (e.g., DNA) segment(s) from one cell to another. The term “vehicle” or “delivery vector” is sometimes used interchangeably with “vector.” It is intended that any form of vehicle or vector be encompassed within this definition. For example, vectors (e.g., viral vectors) include, but are not limited to viral particles, plasmids, transposons, etc.

A first nucleic acid sequence is “operably linked” with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “mutant” includes enzymes which include one or more amino acid variations from the wild-type sequence. For example, a mutant can comprise one or more amino acid additions, deletions or substitutions. In some embodiments, a mutant can be created artificially (for example by site-directed mutagenesis).

Here, the term “homologue” means a protein having a certain homology with the dopamine synthesis enzyme. Here, the term “homology” can be equated with “identity”.

The term “subject” can be used interchangeably with “patient”. Subjects of the present disclosure include but are not limited to human and animal (e.g., pig, cattle, dog, horse, donkey, mouse, hamster, monkeys) subjects.

### Nucleic Acid Construct

Certain aspects of the disclosure relate to a nucleic acid construct comprising nucleotide sequence comprising one, two or three nucleotide sequences of interest (NOIs), each of which encodes an enzyme. The nucleic acid construct can be a DNA or RNA sequence, such as for example a synthetic RNA/DNA sequence, a recombinant RNA/DNA sequence (i.e. prepared by use of recombinant DNA techniques), a cDNA sequence or a partial genomic DNA sequence, including combinations thereof. The present disclosure also encompasses vectors, such as plasmids, comprising a nucleic acid construct of the present disclosure.

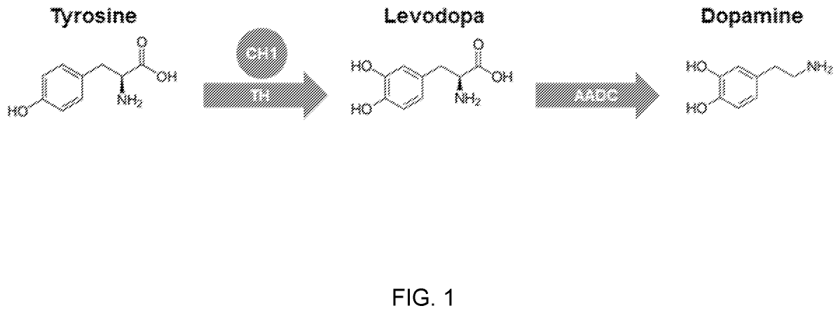

In some embodiments, the NOI in the nucleic acid construct encodes an enzyme involved in dopamine synthesis. A schematic showing endogenous dopamine synthesis including enzymes Tyrosine hydroxylase (TH) and Cyclohydrolase 1 (CH1), which converts tyrosine to levodopa (L-dopa); and Aromatic L-amino acid decarboxylase (AADC), which converts L-dopa to dopamine, is shown in FIG. 1. In some embodiments, the NOI encodes a tyrosine hydroxylase (TH), a GTP-cyclohydrolase I (CH1), an Aromatic Amino Acid Dopa Decarboxylase (AADC), or functional fragments thereof. In some embodiment, the nucleic acid construct comprises a nucleic acid encoding an enzyme selected from the group consisting of a tyrosine hydroxylase (TH), a GTP-cyclohydrolase I (CH1), a Aromatic Amino Acid Dopa Decarboxylase (AADC), or any combination thereof. The sequences of all three full-length enzymes are available: Accession Nos. X05290, U19523 and M76180 respectively.

In some embodiments, NOI can encode all or part of a dopamine synthesis enzyme. For example, the NOI can encode a truncated version of the protein, which retains enzymatic activity. Full length TH comprises a catalytic domain, a tetramerization domain and an N-terminal regulatory domain. In some embodiments, the TH-encoding NOI of the construct of the present disclosure encodes a truncated TH that contains the catalytic and tetramerization domain, but lacks a functional N-terminal regulatory domain. In some embodiments, the truncated TH avoids feedback inhibition by dopamine which may limit activity of the full-length enzyme.

In some embodiments, the NOI can encode a mutant, homologue or variant of the dopamine synthesis enzyme.

In some embodiments, a homologous sequence can be at least 75%, at least 85%, at least 90%, at least 95%, at least 98%, or at least 99% identical to the wild-type or a reference sequence at the amino acid or nucleotide level. In some embodiments, the homologues will comprise or encode the same active sites etc. as the wild-type or reference sequence. Identity comparisons may be conducted, for example, using the BLAST software.

In some embodiments, one or more of the NOI is codon optimized. In some embodiments, one or more of the NOI is not codon optimized.

### Linkers

Certain aspects of the disclosure are directed to a viral vector, e.g., comprising a lentiviral vector genome of the present disclosure, which comprises a nucleic acid construct of the disclosure comprising three NOIs encoding dopamine synthesis enzymes. In some embodiments, at least two of the NOIs are joined by a linker-encoding sequence (L), such that the genome encodes a fusion protein comprising the enzyme amino acid sequences.

In some embodiments, a suitable linker may comprise amino acid repeats such as glycine-serine repeats. The purpose of the linker is to allow the correct formation and/or functioning of the enzymes. It should be sufficiently flexible and sufficiently long to achieve that purpose. Since the NOIs can encode different enzymes, the linker allows the functioning of both of the enzymes. The coding sequence of the flexible linker may be chosen such that it encourages translational pausing and therefore independent folding of the protein products of the NOIs.

In some embodiments, suitable linkers include the linkers disclosed below, but the disclosure is not limited to these particular linkers.

1.
(SEQ ID NO: 2)
(Gly-Gly-Gly-Gly-Ser)₃ as described in
Somia et al., 1993 PNAS 90, 7889.
2.
(SEQ ID NO: 4)
(Gly-Gly-Gly-Gly-Ser)₅.
3
(SEQ ID NO: 5)
(Asn-Phe-Ile-Arg-Gly-Arg-Glu-Asp-Leu-Leu-Glu-Lys-
Ile-Ile-Arg-Gln-Lys-Gl-y-Ser-Ser-Asn) from HSF-
1 of yeast, see Wiederrecht et al.,
1988 Cell 54, 841.
4.
(SEQ ID NO: 6)
(Asn-Leu-Ser-Ser-Asp-Ser-Ser-Leu-Ser-Ser-Pro-Ser-
Ala-Leu-Asn-Ser-Pro-Gl-y-Ile-Glu-Gly-Leu-Ser)
from POU-specific OCT-1, see Dekker
et al., 1993 Nature 362,
852 and Sturm et al., 1988 Genes and Dev. 2, 1582.
5.
(SEQ ID NO: 7)
(Gln-Gly-Ala-Thr-Phe-Ala-Leu-Arg-Gly-Asp-Asn-Pro-
GlnGly) from RGD-containing Laminin peptide,
see Aumailly et al., 1990 FEES Lett.262, 82.
6.
(SEQ ID NO: 8)
(Ser-Gly-Gly-Gly-Glu-Ile-Leu-Asp-Val-Pro-Ser-Thr-
Gly-GlySer-Ser-Pro-Gly) from LDV-containing
linker, see Wickham et al.,
Gene Therapy 1995 2, 750.

In some embodiments, the following GS15 flexible linker can be used: (Gly-Gly-Gly-Gly-Ser)₃. (SEQ ID NO: 2). GS5, GS15, and GS30 linkers may also be suitable.

In some embodiments, the nucleic acid constructs comprise two linkers, e.g., all three enzymes are linked to be expressed as one fusion protein, two non-identical linker-encoding sequences can be chosen, alternatively the linker sequences can be identical. The linker sequences can be identical at the amino acid level, but their encoding nucleic acid sequences can be different due to degeneracy in the genetic code.

In some embodiments, a modified GS15 linker-encoding sequence (GS15 mod) between the TH and CH1 genes is used. In some embodiments, the linker-encoding sequence used in the nucleotide sequence of the present disclosure can be a modified form of a linker-encoding sequence, such as one which encodes GS5, GS15, and GS30, which is not codon optimised for human usage.

In some embodiments, the linker-encoding sequence can comprise the following sequence: GGAGGTGGCGGGTCCGGGGGCGGGGGTAGCGGTGGCGGGGGCTCC (SEQ ID No. 1).

In some embodiments, the nucleotide sequence can encode a linker having the amino acid sequence shown as SEQ ID NO. 2, and the nucleotide sequence can comprise the sequence to that shown in SEQ ID NO. 3. (Gly-Gly-Gly-Gly-Ser)₃ (SEQ ID No. 2); GGGGGAGGCGTAGCGGCGGAGGGGGCTCCGGCGGAGGCGGGAGC (SEQ ID No. 3).

In some embodiments, the construct can comprise the sequence shown as SEQ ID No. 1.

### IRES

When located between open reading frames in an mRNA, an IRES allows translation of the downstream open reading frame by promoting entry of the ribosome at the IRES element followed by downstream initiation of translation. The use of IRES elements in retroviral vectors has been investigated (see, for example, WO 93/0314). Suitable IRES sequences for use in lentiviral vectors are described in WO 02/29065. In some embodiments, the nucleic acid construct of the disclosure comprises an IRES. In some embodiments, the nucleic acid construct comprises TH-_{L}-CH1-_{IRES}-AADC.

### Promoter

In some embodiments, an IRES can be replaced with a promoter, e.g., to control expression of the AADC gene. In configurations where AADC expression is under the control of an IRES, AADC levels may limit dopamine production.

Expression of a NOI can be controlled using control sequences, e.g., promoters/enhancers and other expression regulation signals. Prokaryotic promoters and promoters functional in eukaryotic cells can be used. Tissue specific or stimuli specific promoters can be used. Chimeric promoters can also be used comprising sequence elements from two or more different promoters.

In some embodiments, suitable promoting sequences are strong promoters including those derived from the genomes of viruses—such as polyoma virus, adenovirus, fowlpox virus, bovine papilloma virus, avian sarcoma virus, cytomegalovirus (CMV), retrovirus and Simian Virus 40 (SV40) —or from mammalian cellular promoters—such as the actin promoter or ribosomal protein promoter. Transcription of a gene can be increased further by inserting an enhancer sequence into the vector. Enhancers are relatively orientation and position independent; however, one may employ an enhancer from a eukaryotic cell virus—such as the SV40 enhancer on the late side of the replication origin (bp 100-270) and the CMV early promoter enhancer. The enhancer may be spliced into the vector at a position 5′ or 3′ to the promoter, but is preferably located at a site 5′ from the promoter. In some embodiments, the nucleic acid construct of the disclosure comprises a CMV promoter.

The promoter can additionally include features to ensure or to increase expression in a suitable host. For example, the features can be conserved regions e.g. a Pribnow Box or a TATA box. The promoter can even contain other sequences to affect (such as to maintain, enhance, decrease) the levels of expression of a nucleotide sequence. Suitable other sequences include the Shl-intron or an ADH intron. Other sequences include inducible elements—such as temperature, chemical, light or stress inducible elements. Also, suitable elements to enhance transcription or translation may be present.

In some embodiments, the promoter can, for example, be constitutive or tissue specific.

### Constitutive Promoter

In some examples, suitable constitutive promoters include CMV promoter, RSV promoter, phosphoglycerate kinase (PGK) and thymidine kinase (TK) promoters.

### Tissue Specific Promoter

In some examples, suitable tissue specific promoters include Synapsin 1, Enolase, α-calcium/calmodulin-dependent protein kinase II and GFAP.

### Fusions

In certain embodiments, the viral vector, e.g., a lentiviral vector, comprises a nucleic acid construct of the disclosure comprising a NOIs encoding TH, CHI, and/or AADC. Two of the three, or all three, enzymes can be fused, for example, by using a flexible linker. Where two enzymes are fused the NOI encoding the third enzyme can be operably linked to the nucleotide sequence encoding the fusion protein by, for example, an IRES. The IRES may be positioned 5′ or 3′ to the nucleotide sequence encoding the fusion protein. Alternatively, the NOI encoding the third enzyme can be operatively linked to a promoter.

In some embodiments:

constructs having TH linked to CH1 in that order (i.e. to form a TH-CH1 fusion protein) give high absolute levels of catecholamine production; and

constructs having AADC and TH linked in either order (i.e. to form a AADC-TH or TH-AADC fusion protein) give highly efficient conversion of L-DOPA to dopamine.

In some embodiments, a nucleic acid construct of the present disclosure can encode a fusion of TH and CH1 in the order TC, rather than CT.

In some embodiments, the nucleic acid construct can be selected from the following:

TH-_{L}-CH1-_{IRES}-AADC;
AADC-_{L}-TH-_{L}-CH1;
TH-_{L}-CH1-_{L}-AADC;
TH-_{L}-CH1-_{P}-AADC;
TH-_{L}-ADC-_{IRES}-CH1;
AADC-_{L}-TH-_{IRES}-CH1; and
TH1-_{L}-AADC-_{L}-CH1; wherein
L=linker-encoding sequence

### IRES=Internal Ribosome Entry Site

P=promoter

As mentioned above, wild-type TH comprises a catalytic domain, a tetramerization domain and an N-terminal regulatory domain.

In some embodiments, the TH-encoding NOI can encode a truncated TH that contains the catalytic and tetramerization domain, but which lacks a functional N-terminal regulatory domain.

In some embodiments, the truncated version of TH is fused via a GS15 linker to the CH1.

### Viral Vectors

In some embodiments, the disclosure is directed to use of a viral vector genome, such as a lentiviral vector genome or adeno-associated viral vector genome comprising a nucleotide construct sequence according to the present disclosure. The disclosure also provides a viral vector production system and vector particle comprising such a genome.

In some embodiments, the viral vector of the present disclosure can be derived or derivable from any suitable virus. In some embodiments, the recombinant viral particle is capable of transducing a target cell with a nucleotide sequence of interest (NOI).

For a retroviral particle, once within the cell the RNA genome from the vector particle is reverse transcribed into DNA and integrated into the genome of the target cell.

### Lentiviral Vectors

Lentiviruses are part of a larger group of retroviruses. A detailed list of lentiviruses may be found in Coffin et al. (1997) “Retroviruses” Cold Spring Harbor Laboratory Press Eds: J M Coffin, S M Hughes, H E Varmus pp 758-763). In brief, lentiviruses can be divided into primate and non-primate groups. Examples of primate lentiviruses include but are not limited to: the human immunodeficiency virus (HIV), the causative agent of acquired immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV), feline immunodeficiency virus (FIV) and bovine immunodeficiency virus (BIV).

Lentiviruses differ from other members of the retrovirus family in that lentiviruses have the capability to infect both dividing and non-dividing cells (Lewis et al (1992) EMBO J 11(8):3053-3058) and Lewis and Emerman (1994) J Virol 68 (1):510-516). In contrast, other retroviruses—such as MLV—are unable to infect non-dividing or slowly dividing cells such as those that make up, for example, muscle, eye, brain, lung and liver tissue.

The Equine Infectious Anemia Virus (EIAV) is a member of the lentivirus genus of the retrovirus family. The wild type EIAV virus has a dimeric RNA genome (single-stranded, positive polarity) that is packaged into a spherical enveloped virion containing a nucleoprotein core. Replication of the wild type EIAV genome occurs via reverse transcription and integration into the host cell genome. The genome contains three genes that encode the structural proteins gag, pol, and env, and long terminal repeats (LTR) at each end of the integrated viral genome. In addition to the gag, pol, and env sequence, common to all retroviruses, the EIAV genome contains several short open reading frames (ORFs). These short ORFs are translated from multiply spliced mRNAs. ORF S1 encodes the transcriptional transactivator lat. ORF S2 encodes a protein whose function is unknown, and the ORF S3 appears to encode a rev protein. It is thought that rev is required for the efficient expression of gag, pot and env. Rev acts post-transcriptionally by interacting with an RNA sequence known as the rev-responsive element (RRE), which is located in EIAV within the env gene.

The wild type genome of EIAV also contains several cis-acting sequences, including the R sequence (short repeat at each end of the genome); the U5 sequence (unique sequence element immediately after the R sequence); the U3 sequence (unique sequence element located downstream from the structural proteins); promoter elements that control transcriptional initiation of the integrated provirus; a packaging sequence (herein referred to interchangeably as a packaging site or a packaging signal); and a 5′-splice donor site.

A lentiviral vector, as used herein, can be a vector, e.g., a delivery vector, which comprises at least one component part derivable from a lentivirus. Preferably, that component part is involved in the biological mechanisms by which the vector infects cells, expresses genes or is replicated.

The basic structure of retrovirus and lentivirus genomes share many common features such as a 5′ LTR and a 3′ LTR, between or within which are located a packaging signal to enable the genome to be packaged, a primer binding site, attachment sites to enable integration into a host cell genome and gag, pol and env genes encoding the packaging components—these are polypeptides required for the assembly of viral particles. Lentiviruses have additional features, such as rev and RRE sequences in HIV, which enable the efficient export of RNA transcripts of the integrated provirus from the nucleus to the cytoplasm of an infected target cell.

In the provirus, the viral genes are flanked at both ends by regions called long terminal repeats (LTRs). The LTRs are responsible for transcription by serving as enhancer-promoter sequences and polyadenylation signals thereby controlling the expression of the viral genes.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different viruses.

In a replication-defective lentiviral vector genome gag, pol and env may can be absent or not functional.

In a typical lentiviral vector of the present disclosure, at least part of one or more protein coding regions essential for replication may be removed from the virus. This makes the viral vector replication-defective. Portions of the viral genome may also be replaced by an NOI in order to generate a vector comprising an NOI which is capable of transducing a target non-dividing host cell and/or integrating its genome into a host genome.

In one embodiment the lentiviral vectors are non-integrating vectors such as those disclosed in WO 2007/071994, which is incorporated herein in its entirety.

In some embodiments, the vectors have the ability to deliver a sequence which is devoid of or lacking viral RNA. In a further embodiment a heterologous binding domain (heterologous to gag) located on the RNA to be delivered and a cognate binding domain on gag or pol can be used to ensure packaging of the RNA to be delivered. Both of these vectors are described in WO 2007/072056.

The lentiviral vector can be a “non-primate” vector, i.e., derived from a virus which does not primarily infect primates, especially humans.

In some embodiments, the viral vector can be derived from EIAV. In addition to the gag, pol and env genes EIAV encodes three other genes: tat, rev, and S2. Tat acts as a transcriptional activator of the viral LTR (Derse and Newbold (1993) Virology 194(2):530-536 and Maury et al (1994) Virology 200(2):632-642) and Rev regulates and coordinates the expression of viral genes through rev-response elements (RRE) (Martarano et al. (1994) J Virol 68(5):3102-3111). The mechanisms of action of these two proteins are thought to be broadly similar to the analogous mechanisms in the primate viruses (Martarano et al. (1994) J Virol 68(5):3102-3111). The function of S2 is unknown. In addition, an EIAV protein, Ttm, has been identified that is encoded by the first exon of tat spliced to the env coding sequence at the start of the transmembrane protein (Beisel et al. (1993) J Virol 67(2):832-842).

The term “recombinant lentiviral vector” refers to a vector with sufficient lentiviral genetic information to allow packaging of an RNA genome, in the presence of packaging components, into a viral particle capable of infecting a target cell. Infection of the target cell may include reverse transcription and integration into the target cell genome. The recombinant lentiviral vector carries non-viral coding sequences which are to be delivered by the vector to the target cell. A recombinant lentiviral vector is incapable of independent replication to produce infectious lentiviral particles within the final target cell. Usually the recombinant lentiviral vector lacks a functional gag-pol and/or env gene and/or other genes essential for replication. The vector of the present disclosure may be configured as a split-intron vector. A split intron vector is described in PCT patent application WO 99/15683.

In some embodiments, the recombinant lentiviral vector of the present disclosure may have a minimal viral genome.

As used herein, the term “minimal viral genome” means that the viral vector has been manipulated so as to remove the non-essential elements and to retain the essential elements in order to provide the required functionality to infect, transduce and deliver a nucleotide sequence of interest to a target host cell. Further details of this strategy can be found in our WO 98/17815.

In some aspects of the present disclosure, the vector is a self-inactivating vector. In some aspects, self-inactivating retroviral vectors have been constructed by deleting the transcriptional enhancers or the enhancers and promoter in the U3 region of the 3′ LTR. After a round of vector reverse transcription and integration, these changes are copied into both the 5′ and the 3′ LTRs producing a transcriptionally inactive provirus (Yu et al (1986) Proc. Natl. Acad. Sci. 83:3194-3198; Dougherty and Temin et al (1987) Proc. Natl. Acad. Sci. 84:1197-1201; Hawley (1987) Proc. Natl. Acad. Sci. 84:2406-2410 and Yee et al (1987) Proc. Natl. Acad. Sci. 91:9564-9568). However, any promoter(s) internal to the LTRs in such vectors will still be transcriptionally active. This strategy has been employed to eliminate effects of the enhancers and promoters in the viral LTRs on transcription from internally placed genes. Such effects include increased transcription (Jolly et al (1983) Nucleic Acids Res. 11:1855-1872) or suppression of transcription (Emerman and Temin (1984) Cell 39:449-467). This strategy can also be used to eliminate downstream transcription from the 3′ LTR into genomic DNA (Herman and Coffin (1987) Science 236:845-848). This is of particular concern in human gene therapy where it is of critical importance to prevent the adventitious activation of an endogenous oncogene.

However, the plasmid vector used to produce the viral genome within a host cell/packaging cell will also include transcriptional regulatory control sequences operably linked to the lentiviral genome to direct transcription of the genome in a host cell/packaging cell. These regulatory sequences may be the natural sequences associated with the transcribed lentiviral sequence, i.e. the 5′ U3 region, or they may be a heterologous promoter such as another viral promoter, for example the CMV promoter. Some lentiviral genomes require additional sequences for efficient virus production. For example, in the case of HIV, rev and RRE sequence are preferably included. However the requirement for rev and RRE may be reduced or eliminated by codon optimisation of gag-pol (as described in WO 01/79518) and/or the inclusion of an Open Reading Frame downstream of the LTR and upstream of the internal promoter (as described in WO 03/064665), for example neo has been used in certain constructs disclosed herein, however, the skilled person could use any suitable Open Reading Frame. Alternative sequences, which perform the same function as the rev/RRE system, are also known. For example, a functional analogue of the rev/RRE system is found in the Mason Pfizer monkey virus. This element is known as the constitutive transport element (CTE), and comprises an RRE-type sequence in the genome, which is believed to interact with a factor in the infected cell. The cellular factor can be thought of as a rev analogue. Thus, CTE may be used as an alternative to the rev/RRE system. Any other functional equivalents, which are known or become available, may be relevant to the disclosure. For example, it is also known that the Rex protein of HTLV-I can functionally replace the Rev protein of HIV-1. It is also known that Rev and Rex have similar effects to IRE-BP.

The lentiviral vector according to the present disclosure may comprise of a self-inactivating minimal lentiviral vector, derived from Equine Infectious Anaemia Virus (EIAV), preferably encoding three enzymes that are involved in the dopamine synthetic pathway. The proteins encoded by such a vector may comprise a truncated form of the human tyrosine hydroxylase gene (which lacks the N-terminal 160 amino acids involved in feedback regulation of TH), the human aromatic L-amino-acid decarboxylase (AADC), and the human GTP-cyclohydrolase 1 (GTP-CH1) gene. The vector can be produced by the transient transfection of cells (e.g. HEK293T cells) with three plasmids, encoding for: (1) the vector genomes as described herein (2) the synthetic EIAV gag/pol expression vector (pESGPK, WO 01/79518 and WO 05/29065) and (3) the VSV-G envelope expression vector (pHGK).

### Packaging Sequence

The term “packaging signal” which is referred to interchangeably as “packaging sequence” or “psi” is used in reference to the non-coding, cis-acting sequence required for encapsidation of lentiviral RNA strands during viral particle formation. In HIV-1, this sequence has been mapped to loci extending from upstream of the major splice donor site (SD) to at least the gag start codon.

As used herein, the term “extended packaging signal” or “extended packaging sequence” refers to the use of sequences around the psi sequence with further extension into the gag gene. The inclusion of these additional packaging sequences may increase the efficiency of insertion of vector RNA into viral particles.

### Pseudotyping

In some embodiments, the lentiviral vector of the present disclosure is pseudotyped. In this regard, pseudotyping can confer one or more advantages. For example, with the lentiviral vectors, the env gene product of the HIV based vectors would restrict these vectors to infecting only cells that express a protein called CD4. But, if the env gene in these vectors has been substituted with env sequences from other viruses, then they may have a broader infectious spectrum (Verma and Somia (1997) Nature 389(6648):239-242). By way of examples, Miller et al. pseudotyped a MoMLV vector with the envelope from the amphotropic retrovirus 4070A (Mol. Cell. Biol. 5:431-437). Others have pseudotyped an HIV based lentiviral vector with the glycoprotein from VSV (Verma and Somia (1997) Nature 389(6648):239-242).

In some embodiments, the Env protein is a modified Env protein such as a mutant or engineered Env protein. Modifications can be made or selected to introduce targeting ability or to reduce toxicity or for another purpose (Marin et al (1996) J Virol 70(5):2957-2962; Nilson et al (1996) Gene Ther 3(4):280-286; and Fielding et al (1998) Blood 91(5):1802-1809 and references cited therein).

In some embodiments, the vector can be pseudotyped, for example with a gene encoding at least part of the rabies G protein or the VSV-G protein.

### VSV-G

In some embodiments, the envelope glycoprotein (G) of Vesicular stomatitis virus (VSV), a rhabdovirus, is an envelope protein that has been shown to be capable of pseudotyping certain retroviruses including lentiviruses.

Its ability to pseudotype MoMLV-based retroviral vectors in the absence of any retroviral envelope proteins was first shown by Emi et al. (1991) J. Virol. 65:1202-1207). WO 94/294440 teaches that retroviral vectors may be successfully pseudotyped with VSV-G. These pseudotyped VSV-G vectors may be used to transduce a wide range of mammalian cells. More recently, Abe et al. (1998) J. Virol 72(8): 6356-6361 teach that non-infectious retroviral particles can be made infectious by the addition of VSV-G.

Burns et al (1993) Proc. Natl. Acad. Sci. USA 90:8033-8037) successfully pseudotyped the retrovirus MLV with VSV-G and this resulted in a vector having an altered host range compared to MLV in its native form. VSV-G pseudotyped vectors have been shown to infect not only mammalian cells, but also cell lines derived from fish, reptiles and insects (Burns et al (1993) Proc. Natl. Acad. Sci. USA 90:8033-8037). They have also been shown to be more efficient than traditional amphotropic envelopes for a variety of cell lines (Yee et al. (1994) Proc. Natl. Acad. Sci. USA 91:9564-9568 and Emi et al. (1991) J. Virol. 65:1202-1207). VSV-G protein can also be used to pseudotype certain lentiviruses and retroviruses because its cytoplasmic tail is capable of interacting with the retroviral cores.

The provision of a non-lentiviral pseudotyping envelope such as VSV-G protein gives the advantage that vector particles can be concentrated to a high titre without loss of infectivity (Akkina et al (1996) J. Virol. 70:2581-2585). Lentivirus and retrovirus envelope proteins are apparently unable to withstand the shearing forces during ultracentrifugation, probably because they consist of two non-covalently linked subunits. The interaction between the subunits may be disrupted by the centrifugation. In comparison the VSV glycoprotein is composed of a single unit. VSV-G protein pseudotyping can therefore offer potential advantages.

WO 00/52188 describes the generation of pseudotyped retroviral and lentiviral vectors, from stable producer cell lines, having vesicular stomatitis virus-G protein (VSV-G) as the membrane-associated viral envelope protein, and provides a gene sequence for the VSV-G protein.

### Ross River Virus

The Ross River viral envelope has been used to pseudotype a non-primate lentiviral vector (FIV) and following systemic administration predominantly transduced the liver (Kang et al (2002) J Virol 76(18):9378-9388). Efficiency was reported to be 20-fold greater than obtained with VSV-G pseudotyped vector, and caused less cytotoxicity as measured by serum levels of liver enzymes suggestive of hepatotoxicity.

Ross River Virus (RRV) is an alphavirus spread by mosquitoes which is endemic and epidemic in tropical and temperate regions of Australia. Antibody rates in normal populations in the temperate coastal zone tend to be low (6% to 15%) although sero-prevalence reaches 27 to 37% in the plains of the Murray Valley River system. In 1979 to 1980 Ross River Virus became epidemic in the Pacific Islands. The disease is not contagious between humans and is never fatal, the first symptom being joint pain with fatigue and lethargy in about half of patients (Fields Virology Fifth Edition (2007) Eds. Knipe and Howley. Lippincott Williams and Wilkins)

### Baculovirus GP64

The baculovirus GP64 protein has been shown to be an attractive alternative to VSV-G for viral vectors used in the large-scale production of high-titre virus required for clinical and commercial applications (Kumar M, Bradow B P, Zimmerberg J (2003) Hum. Gene Ther. 14(1):67-77). Compared with VSV-G-pseudotyped vectors, GP64-pseudotyped vectors have a similar broad tropism and similar native titres. Because, GP64 expression does not kill cells, 293T-based cell lines constitutively expressing GP64 can be generated.

### Rabies G

In the present disclosure the vector may be pseudotyped with at least a part of a rabies G protein or a mutant, variant, homologue or fragment thereof.

Teachings on the rabies G protein, as well as mutants thereof, may be found in WO 99/61639 and well as Rose et al (1982) J. Virol. 43:361-364, Hanham et al (1993) J. Virol. 67:530-542; Tuffereau et al (1998) J. Virol. 72:1085-1091, Kucera et al (1985) J. Virol. 55:158-162; Dietzschold et al (1983) PNAS 80:70-74; Seif et al (1985) J. Virol. 53:926-934; Coulon et al (1998) J. Virol. 72:273-278; Tuffereau et al (1998) J. Virol. 72:1085-10910; Burger et al (1991) J. Gen. Virol. 72:359-367; Gaudin et al (1995) J. Virol. 69:5528-5534; Benmansour et al (1991) J. Virol. 65:4198-4203; Luo et al (1998) Microbiol. Immunol. 42:187-193, Coll (1997) Arch. Virol. 142:2089-2097; Luo et al (1997) Virus Res. 51:35-41; Luo et al (1998) Microbiol. Immunol. 42:187-193; Coll (1995) Arch. Virol. 140:827-851; Tuchiya et al (1992) Virus Res. 25:1-13; Morimoto et al (1992) Virology 189:203-216; Gaudin et al (1992) Virology 187:627-632; Whitt et al (1991) Virology 185:681-688; Dietzschold et al (1978) J. Gen. Virol. 40:131-139; Dietzschold et al (1978) Dev. Biol. Stand. 40:45-55; Dietzschold et al (1977) J. Virol. 23:286-293 and Otvos et al (1994) Biochim. Biophys. Acta 1224:68-76. A rabies G protein is also described in EP 0445625.

### Alternative Envelopes

Other envelopes which can be used to pseudotype lentiviral vectors include Mokola, Ebola, 4070A and LCMV (lymphocytic choriomeningitis virus).

### Adeno-Associated Viral Vectors

It had been known in the art that adeno-associated viral (AAV) vectors have limited packaging capacity therefore negatively impacting the number of genes that can be delivered efficiently. However, it is now known that this limitation is dependent on AAV serotype. For instance, capsids of AAV5 and AAV7 serotypes can package genomes of up to 8 kb. This work has been described in U.S. Pat. No. 7,943,374. In addition, US 2009/0214478 describe AAV2/5 recombinant vectors with a packaging capacity up to 9 kb.

Features of AAV vectors are generally known to one of ordinary skill in the art. For example, AAV vectors have a broad host range and transduce both dividing and non-dividing cells with relatively low immunogenicity. It is also well known how to replace all AAV viral genes with a genetic cassette leaving in place only cis-acting AAV elements the Inverted Terminal Repeats (ITRs), the DNA packaging signal, and the replication origin. See e.g., Musatov et al., J. Virol., December 2002, 76(24). AAV can be packaged in producer cells when AAV gene products, Rep and Cap, and other accessory proteins are provided in trans. AAV packaging systems have been described. See, e.g., U.S. Pat. No. 5,139,941. Non-AAV accessory functions may be supplied by any of the known helper viruses such as Adenovirus, Herpes Simplex Virus, and vaccinia virus. Such AAV packaging systems have been described; e.g., in U.S. Pat. Nos. 4,797,368; 5,139,941; 5,866,552; 6,001,650; 6,723,551.

### Codon Optimization

In some embodiments, the polynucleotides used in the present disclosure (including all or portions of the nucleic acid constructs, NOI and/or vector components) can be codon optimized. Codon optimization has previously been described in WO 99/41397 and WO 01/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

Many viruses, including HIV and other lentiviruses, use a large number of rare codons and by changing these to correspond to commonly used mammalian codons, increased expression of a gene of interest, e.g. a NOI or packaging components in mammalian producer cells, can be achieved. Codon usage tables are known in the art for mammalian cells, as well as for a variety of other organisms.

Codon optimization of viral vector components has a number of other advantages. By virtue of alterations in their sequences, the nucleotide sequences encoding the packaging components of the viral particles required for assembly of viral particles in the producer cells/packaging cells have RNA instability sequences (INS) eliminated from them. At the same time, the amino acid sequence coding sequence for the packaging components is retained so that the viral components encoded by the sequences remain the same, or at least sufficiently similar that the function of the packaging components is not compromised. In lentiviral vectors codon optimization also overcomes the Rev/RRE requirement for export, rendering optimized sequences Rev independent. Codon optimization also reduces homologous recombination between different constructs within the vector system (for example between the regions of overlap in the gag-pol and env open reading frames). In some embodiments, the overall effect of codon optimization is a notable increase in viral titre and improved safety.

In some embodiment only codons relating to INS are codon optimized. However, in some embodiments, the sequences are codon optimized in their entirety, with some exceptions, for example the sequence encompassing the frameshift site of gag-pol (see below).

The gag-pol gene comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling due to RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimised. Retaining this fragment will enable more efficient expression of the gag-pol proteins.

For EIAV, the beginning of the overlap has been taken to be nt 1262 (where nucleotide 1 is the A of the gag ATG). The end of the overlap is at 1461 bp. In order to ensure that the frameshift site and the gag-pol overlap are preserved, the wild type sequence has been retained from nt 1156 to 1465.

Derivations from optimal codon usage may be made, for example, in order to accommodate convenient restriction sites, and conservative amino acid changes may be introduced into the gag-pol proteins.

In one embodiment codon optimization is based on lightly expressed mammalian genes. The third, and sometimes the second and third base of each codon may be changed.

Due to the degenerate nature of the Genetic Code, it will be appreciated that numerous gag-pol sequences can be achieved by a skilled worker. Also, there are many retroviral variants described which can be used as a starting point for generating a codon optimised gag-pol sequence. Lentiviral genomes can be quite variable. For example, there are many quasi-species of HIV-1 which are still functional. This is also the case for EIAV. These variants may be used to enhance particular parts of the transduction process. Examples of HIV-1 variants may be found at the HIV Databases operated by Los Alamos National Security. Details of EIAV clones may be found at the National Center for Biotechnology Information (NCBI) database.

The strategy for codon optimized gag-pol sequences can be used in relation to any retrovirus. This would apply to all lentiviruses, including EIAV, FIV, BIV, CAEV, VMR, SIV, HIV-1 and HIV-2. In addition, this method could be used to increase expression of genes from HTLV-1, HTLV-2, HFV, HSRV and human endogenous retroviruses (HERV), MLV and other retroviruses.

Codon optimization can render gag-pol expression Rev independent. In order to enable the use of anti-rev or RRE factors in the lentiviral vector, however, it would be necessary to render the viral vector generation system totally Rev/RRE independent. Thus, the genome also needs to be modified. This is achieved by optimizing vector genome components. Advantageously, these modifications also lead to the production of a safer system absent of all additional proteins both in the producer and in the transduced cell.

### Activity

In certain aspects of the disclosure, the fusion constructs of the disclosure produce functional dopaminergic synthesizing enzymes and can cause an increase in dopamine production when compared to the levels obtained using a construct with all three genes encoding the dopamine synthesizing enzymes separated by IRES sequences described in WO 02/29065.

In some embodiments, the vector of the present disclosure can cause increased L-DOPA and/or dopamine production when expressed intracellularly than that of the vector described in WO 2001/04433, pONYK1.

The vector of the present disclosure can give at least 2, 3, 5, 10, 15, 20, 30, 40, 50, 60, 80, 80, 90, 100, 120, 130, 140, 150, 160, 200, 500, 1000-fold increase in dopamine and/or L-DOPA production.

The vector of the present disclosure can cause increased L-DOPA and/or dopamine production when compared to pONYK1 when expressed, for example, in HEK293T cells or PC-12 cells.

Dopamine or L-DOPA production can be measured by any of a number of methods known in the art, such as high performance liquid chromatography (HPLC).

Without wishing to be bound by theory, the present inventors suggest that L-DOPA and/or dopamine synthesis is increased by the fusion protein because the encoded proteins are physically close together, thereby facilitating their interactions with one another. This is particularly advantageous for enzymes of the dopamine biosynthetic pathway as physical proximity of each of the enzymes may facilitate efficient metabolite flow from one enzyme to the other enabling maximal L-DOPA or dopamine production.

In some embodiments, the fusion construct of the disclosure provide improved L-DOPA production and improved dopamine production compared to baseline, and in some embodiments compared other constructs such as ProSavin®.

### Pharmaceutical Compositions and Dosing

The viral vector, e.g., lentiviral vector, comprising a nucleic acid construct, of the present disclosure (e.g., Lenti-PD) can be provided in the form of a pharmaceutical composition. The pharmaceutical composition can be used for treating an individual by gene therapy, wherein the composition comprises a therapeutically effective amount of the viral vector, e.g., a lentiviral vector, comprising a construct of the present disclosure (e.g., Lenti-PD).

In some embodiments, the viral preparation can be concentrated by ultracentrifugation. In some embodiments, the lentiviral vector can be can be processed according to any of the methods disclosed in WO 2009/153563, which is incorporated herein by reference in its entirety.

A therapeutically effective amount of a viral vector is an amount sufficient to deliver a nucleic acid construct of the disclosure to a target cell population or target tissue such that the viral vector provides a clinically relevant effect. The effective amount can depend on factors such as the species, age, weight, health of the subject, and the tissue to be targeted, and may thus vary among animals and tissues. The pharmaceutical composition can be used to treat a human subject. In some embodiments, a subject is suffering from a neurodegenerative disease or a disease where dopamine levels are reduced in the subject. In some embodiments, the human suffers from Parkinson's Disease, e.g., Bilateral Idiopathic Parkinson's Disease.

In some embodiments, the pharmaceutical composition comprises a viral vector, e.g., lentiviral particles, of the disclosure. In some embodiments, the viral vector dose can comprise least 10⁶ transducing units (TU), for example 1×10⁶ TU to 5×10⁸ TU, inclusive. The TU can be calculated based on the number of integration events, e.g., using an integration (DNA) titer assay. The vector titer can be expressed in TU per mL (TU/mL) for vector strength as titred on the standard HEK293T cell line, or as a target dose delivered to the subject (TU/subject) (e.g., calculated relative to the target strength of the vector).

In some embodiments, the target dose comprises 2×10⁶ TU/subject to 2×10⁸ TU/subject; 3×10⁶ TU/subject to 2×10⁸ TU/subject; 4×10⁶ TU/subject to 2×10⁸ TU/subject, 5×10⁶ TU/subject to 2×10⁸ TU/subject, 6×10⁶ TU/subject to 2×10⁸ TU/subject, 7×10⁶ TU/subject to 2×10⁸ TU/subject, 8×10⁶ TU/subject to 2×10⁸ TU/subject, or 9×10⁶ TU/subject to 2×10⁸ TU/subject.

In some embodiments, the target dose comprises 1×10⁷ TU/subject to 1×10⁸ TU/subject; 2×10⁷ TU/subject to 1×10⁸ TU/subject; 3×10⁷ TU/subject to 1×10⁸ TU/subject; 4×10⁷ TU/subject to 1×10⁸ TU/subject, 5×10⁷ TU/subject to 1×10⁸ TU/subject, 6×10⁷ TU/subject to 1×10⁸ TU/subject, 7×10⁷ TU/subject to 1×10⁸ TU/subject, 8×10⁷ TU/subject to 1×10⁸ TU/subject, or 9×10⁷ TU/subject to 1×10⁸ TU/subject.

In some embodiments, the total target dose comprises 1×10⁷ TU/subject to 2×10⁷ TU/subject, 2×10⁷ TU/subject to 3×10⁷ TU/subject, 3×10⁷ TU/subject to 4×10⁷ TU/subject, 4×10⁷ TU/subject to 5×10⁷ TU/subject, 5×10⁷ TU/subject to 6×10⁷ TU/subject, 6×10⁷ TU/subject to 7×10⁷ TU/subject, 7×10⁷ TU/subject to 8×10⁷ TU/subject, 8×10⁷ TU/subject to 9×10⁷ TU/subject, or 9×10⁷ TU/subject to 1×10⁸ TU/subject.

In some embodiments, the target dose is about 1×10⁶ TU/subject to 5×10⁸ TU/subject, 4×10⁶ TU/subject to 8×10⁶ TU/subject; 8×10⁶ TU/subject to 4×10⁷ TU/subject; or 1×10⁷ TU/subject to 5×10⁸ TU/subject. In some embodiments, the target dose is about 1.5×10⁷ TU/mL to 5×10⁷ TU/mL.

In some embodiments, the target dose is about 2×10⁶ TU/subject, about 1.4×10⁷ TU/subject, about 1.5×10⁷ TU/subject, about 1.6×10⁷ TU/subject, about 1.7×10⁷ TU/subject, about 1.8×10⁷ TU/subject, about 1.9×10⁷ TU/subject, about 2×10⁷ TU/subject, about 2.1×10⁷ TU/subject, about 2.2×10⁷ TU/subject, about 2.3×10⁷ TU/subject, about 2.4×10⁷ TU/subject, or about 2.5×10⁷ TU/subject.

In some embodiments, the target vector dose strength comprises 5×10⁶ TU/mL to 5×10⁸ TU/mL, 5×10⁶ TU/mL to 5×10⁷ TU/mL, 1×10⁷ TU/mL to 5×10⁷ TU/mL, 1.5×10⁷ TU/mL to 5×10⁷ TU/mL, or 5×10⁷ TU/mL to 5×10⁸ TU/mL.

In some embodiments, the total target vector dose is administered to the subject in multiple small volumes (e.g., 50-200 μL) at a controlled rate (e.g., about 2-4 μL/minute) directly into the putamen.

The composition may optionally comprise a pharmaceutically acceptable carrier, excipient, or diluent. The choice of pharmaceutical carrier, excipient or diluent can be selected with regard to the intended route of administration and standard pharmaceutical practice. The pharmaceutical compositions may comprise as (or in addition to) the carrier, excipient or diluent, any suitable binder(s), lubricant(s), suspending agent(s), coating agent(s), solubilising agent(s), and other carrier agents that may aid or increase the viral entry into the target site. In some embodiments, the pharmaceutical composition of the disclosure comprises an excipient selected from the group consisting of a tonicity agent, a cryopreservation aid, a pH buffering agent, a pH adjusting agent, or any combination thereof. In some embodiments, the tonicity agent comprises sodium chloride. In some embodiments, the cryopreservation aid comprises mannitol and/or sucrose. In some embodiments, the pH buffering agent comprises Tromethamine (TRIS). In some embodiments, the pH adjusting agent comprises Hydrochloric acid (HCl). In some embodiments, the diluent is water.

### Administration

In some embodiments, the viral vector comprising a construct of the present disclosure (e.g., Lenti-PD) is administered to the brain, for example, by injection into the caudate putamen. In some embodiments, the viral vector is administered by injection to the sensorimotor putamen. In some embodiments, the vector is continuously infused to the brain, e.g., according to any of the methods disclosed in U.S. Pat. No. 9,339,512, which is incorporated herein by reference in its entirety.

In some embodiments, the viral vector is administered by local bilateral stereotactic infusion into the sensorimotor putamen of PD subjects allowing for expression of the encoded proteins in non-dopaminergic neurons. In some embodiments, the administration is in multiple small volumes (e.g., 50-200 μL) at a controlled rate (about 2-4 μL/minute, e.g., 3 μL/minute) directly into the putamen made via 1-6, e.g., three, tracts in the first hemisphere and 1-6, e.g., three, tracts in the second hemisphere.

The viral vector can be administered via one, two, three, four, five, six or more tracts per hemisphere.

In some embodiments, the viral vector can be administered as a previously described administration system for a lentiviral vector (Jarraya et al (2009) Sci Transl Med 14: 1(2) 2-4), incorporated herein by reference in its entirety. For example, the viral vector composition can administered in a discontinuous or “punctate” fashion, by administering an aliquot (2-4 μL) at the bottom of the tract, withdrawing the needle a little way, then administering a second aliquot (2-4 μL) and withdrawing the needle a little further, (second time); then administering a third aliquot (2-4 μL); thus aliquots had been deposited at 3 points along each needle tract delivering a total of about 10 μL.

### Methods of Use

Certain aspects of the disclosure are directed to methods of providing one or more of the following to a subject suffering from Parkinson's Disease: (a) improving motor function, (b) reducing dyskinesia, and/or (c) allowing for a reduction in the subject's daily dose of L-DOPA or LED, wherein the method comprises administering a viral vector comprising a nucleic acid construct disclosed herein (e.g., Lenti-PD) to a subject in need thereof.

Certain aspects of the disclosure are directed to methods of improving motor function and/or reducing dyskinesia in a subject suffering therefrom comprising administering a viral vector comprising a construct disclosed herein.

One of the main issues affecting the pharmacological treatment of PD is deciding when to initiate L-dopa therapy. Early administration has been shown to confer the greatest clinical benefits, although it is known that long-term treatment with L-dopa is associated with unpleasant motor side effects. Consequently, many physicians prefer to delay implementation of L-dopa as initial therapy. However, L-dopa induces dyskinesia as a side effect.

In some embodiments, the subject is suffering from a neurodegenerative disease or a disease where endogenous dopamine levels are reduced in the subject. In some embodiments, the subject has Parkinson's Disease. In some embodiments, the subject is undergoing L-DOPA (also known as levodopa) therapy. In some embodiments, the subject is undergoing levodopa equivalent dose (LED) therapy. In some embodiments, the subject is also administered carbidopa.

In some embodiments, when administered to a patient undergoing L-Dopa or LED therapy, a viral vector comprising a nucleic acid construct disclosed herein (e.g., Lenti-PD) allows the daily dosage of L-dopa to be reduced while improving the tapering of symptoms and reducing dyskinesia associated with administration of L-dopa or LED therapy. The combined treatment produces improvement in motor functionality of Parkinson's disease patients, and also improves motor fluctuations induced by pharmacological therapy. The combination, specifically decreases dyskinesias, the “on-off” and the wearing-off phenomenon. In some embodiments, the reduction in dyskinesia it determined based on the rating scale from baseline using the Rush Dyskinesia Rating Scale (RDRS) in “OFF” and “ON” states.

In some embodiments, dyskinesias is reduced in the subject as shown by one or more of the following: (a) at least 50%, at least 60%, at least 70%, at least 75%, at least 80%, at least 85%, or at least 90% reduction in Hauser diary ON time with troublesome dyskinesia 3 months after administration compared to baseline; (b) at least 50% reduction, at least 60%, at least 65%, at least 70%, at least 74%, or at least 75% reduction in UPDRS-IV score 3 months after administration compared to baseline, and (c) at least 10%, at least 12%, at least 15%, at least 18%, at least 20%, or at least 25% improvement in Rush Dyskinesia score 3 months after administration compared to baseline.

In some embodiments, clinical standardized measurements for motor function, e.g., the UPDRS-III (motor) OFF score is improved after the administration of the viral vector comprising the nucleic acid construct of the disclosure (e.g., Lenti-PD). In some embodiments, the motor function is improved as shown by at least 25%, at least 30%, at least 35%, at least 40%, at least 42%, at least 45%, or at least 50% increase in average UPDRS-III (motor) OFF score 3 months after administration compared to baseline. In some embodiments, the motor function is improved as shown by at least 12, at least 15, at least 20, at least 22, at least 25, at least 30, or at least 35 point increase in average UPDRS-III (motor) OFF score 3 months after administration compared to baseline.

In some embodiments, about three months after administration, the subject's average UPDRS OFF total score has improved by at least 25%, at least 30%, at least 35%, at least 40%, at least 45%, at least 55%, or at least 60% from baseline. In some embodiments, the subject's baseline UPDRS Part III (motor) OFF score is between 30-60.

In some embodiments, the UPDRS-II (activities of daily living) OFF score is improved after the administration of the viral vector comprising the nucleic acid construct of the disclosure (e.g., Lenti-PD). In some embodiments, the UPDRS-II OFF score is improved as shown by at least 25%, at least 30%, at least 35%, at least 40%, at least 42%, at least 45%, at least 50%, at least 60%, at least 65%, at least 70%, at least 75%, or at least 80% increase in average UPDRS-II OFF score 3 months after administration compared to baseline. In some embodiments, the motor function is improved as shown by at least 12, at least 15, at least 20, at least 22, at least 25, at least 30, or at least 35 point increase in average UPDRS-II OFF score 3 months after administration compared to baseline.

In some embodiments, the UPDRS-IV (complications of therapy) OFF score is improved after the administration of the viral vector comprising the nucleic acid construct of the disclosure (e.g., Lenti-PD). In some embodiments, the UPDRS-IV OFF score is improved as shown by at least 25%, at least 30%, at least 35%, at least 40%, at least 42%, at least 45%, at least 50%, at least 60%, at least 65%, at least 70%, at least 75%, or at least 80% increase in average UPDRS-IV OFF score 3 months after administration compared to baseline. In some embodiments, the motor function is improved as shown by at least 5, at least 6, at least 7, at least 8, at least 9, at least 10, or at least 12 point increase in average UPDRS-II OFF score 3 months after administration compared to baseline.

In some embodiments, the viral vector of the disclosure can be used for treating a neurological condition. For example, the vector can be useful for the treatment and/or prevention of neurodegenerative diseases such as Parkinson's Disease (PD). The disease may be treatable by the production of L-DOPA and/or dopamine in a subject. The disease may be Parkinson's disease, e.g., Bilateral Idiopathic Parkinson's Disease. In some embodiments, the treatment is for the late stages of PD, e.g., in patients who have become refractory to oral L-DOPA treatment.

Certain constructs described herein increase the production of L-DOPA and/or increase the production of dopamine. Increased production of L-DOPA can be useful in patients who retain residual AADC enzymatic activity and are thus at least partly capable of converting L-DOPA to dopamine. These patients can be susceptible to conventional L-DOPA treatment. For example, TH-_{L}-CH1-_{IRES}-AADC constructs produced high levels of both dopamine and L-DOPA, whereas TH-_{L}-AADC-_{IRES}-CH1, and AADC-_{L}-TH-_{L}-CH1 produced higher levels of dopamine relative to L-DOPA.

Increased production of dopamine can be useful in late-stage patients who lack sufficient endogenous AADC activity to process L-DOPA, and are thus less sensitive to conventional L-DOPA treatment.

In some embodiments, the subject is on L-DOPA or LED therapy (LED=L-DOPA equivalent dose of drugs). In some embodiments, the subject is undergoing L-DOPA or LED therapy. In some embodiments, the subject's L-DOPA or LED therapy dose is reduced after administration of a viral vector comprising a nucleic acid construction of the disclosure relative to the subject's L-DOPA or LED therapy dose prior to administration of the nucleic acid construct. In some embodiments, the average daily L-DOPA or LED therapy dose is reduced by at least 10%, at least 12%, at least 14%, at least 15%, at least 16%, at least 17%, at least 18%, at least 19%, at least 20%, or at least 25% from baseline three months after administration of a viral vector comprising a nucleic acid construction of the disclosure.

In some embodiments, the methods of the disclosure improve one or more symptoms of Parkinson's Disease. In some embodiments, the one or more symptoms are selected from the group consisting of tremors, bradykinesia, rigid muscles, impaired posture and/or balance, loss of automatic movements, difficulty speaking and difficulty with fine motor skills. In some embodiments, the symptom includes tremors, or shaking, usually begins in a limb, often in hands or fingers. In some embodiments, the symptoms include bradykinesia (slowed movement). Over time, Parkinson's disease may slow a subject's movement, making simple tasks difficult and time-consuming. In some embodiments, the symptoms include rigid muscles, e.g., muscle stiffness in any part of a subject's body. The stiff muscles can be painful and limit range of motion. In some embodiments, the symptoms include impaired posture and/or balance. In some embodiments, the symptoms include difficulties speaking. In some embodiments, the symptoms include difficulty with fine motor skills, e.g., writing.

The disclosure will now be further described by way of Examples, which are meant to serve to assist one of ordinary skill in the art in carrying out the disclosure and are not intended in any way to limit the scope of the invention.

### EXAMPLES

### Example 1

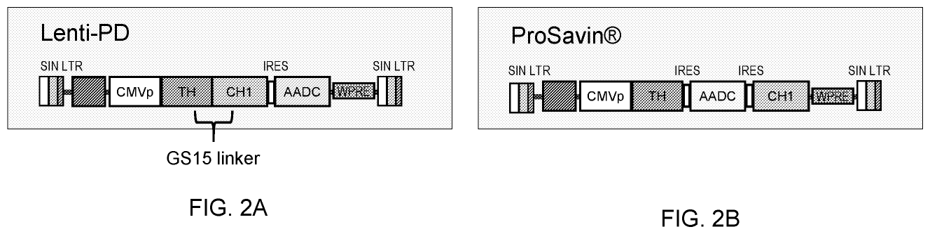

“Lenti-PD” is a non-replicating non-primate recombinant lentiviral vector (LV) based on the non-pathogenic wild type equine infectious anaemia virus (EIAV). The wild type EIAV genome has 6 distinct genetic units, however, the majority of these EIAV sequences (˜90%) have been removed to produce a replication defective minimal vector system that contains less than 10% of the original viral genome and does not contain any of the natural viral promoters or enhancers and there are no coding regions for accessory proteins in either the EIAV genome or in the packaging system. Lenti-PD contains a dopamine enzyme fusion plasmid encoding the truncated form of human tyrosine hydroxylase (TH), human GTP-cyclohydrolase 1 (CH1), and human aromatic L-amino-acid decarboxylase (AADC). The Lenti-PD construct is shown in FIG. 2A.

ProSavin® is an Equine Infectious Anaemia Virus (EIAV) based LV. ProSavin® also contains a tricistronic construct comprising the coding sequences TH, AADC and CH1, which in this construct are operably linked by two internal ribosome entry sites (IRES). The construct for ProSavin® is shown in FIG. 2B.

Differences between the ProSavin® and Lenti-PD constructions include that in Lenti-PD, CH1 is moved closer to the promoter to enhance expression, and in Lenti-PD, TH and CH1 are joined by a flexible linker to ensure co-localization.

To prepare the plasmids, a minimal pONYK1 genome plasmid, more recently described as pONY8.9.4TY (containing the KanR gene) (Jarraya et al., 2009 Science Translational Medicine 1, 2ra4) was used. This plasmid was based on pONY8.0T, which is described in greater detail by Azzouz M et al (Azzouz et al., 2002 J Neurosci 22, 10302-10312). In brief, pONYK1 is an EIAV SIN vector genome into which was inserted a cassette containing (in order): Neo, an internal CMV promoter, truncated, codon-optimised human tyrosine hydroxylase (TH), EMCV internal ribosome entry site (IRES), codon-optimised human aromatic amino acid dopa decarboxylase (AADC), poliovirus IRES, GTP-cyclohydrolase I (GTP-CH1), and the woodchuck hepatitis virus post-transcriptional regulatory element (WPRE). The Lenti-PD fusion plasmid containing two fused genes (TH and CH1) and one PV IRES element was generated by inserting regions of synthesised DNA (GeneArt, Germany) into the tricistronic cassette replacing the EMCV IRES region and the stop codon of the first gene with a GS15 linker. The GS15 linker codes for 4× glycine amino acids followed by 1× serine amino acid repeated in triplicate, which yields a linker of fifteen amino acids. The DNA sequence for this GS15 linker is as follows:

(SEQ ID NO: 3)
GGGGGAGGCGGTAGCGGCGGAGGGGGCTCCGGCGGAGGCGGGAGC.

The VSV-G envelope and EIAV synthetic Gag/Pol plasmids were used for viral vector production in HEK293T cells. The vector titres, in transducing units/ml (TU/ml), were estimated by integration (DNA) titre assay.

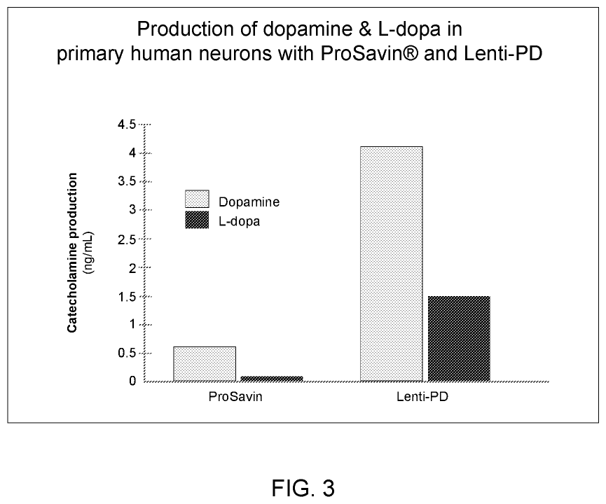

Lenti-PD achieved a larger increase in production of dopamine and L-dopa in primary human neurons in vitro compared to ProSavin® (FIG. 3).

### Example 2

### Phase 2 Parkinson's Disease Study (Low Dose—Cohort 1)

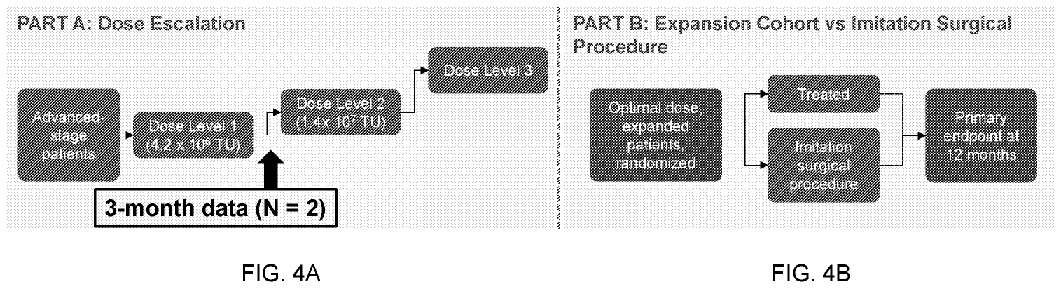

A phase 2 study (SUNRISE-PD, NCT03720418) of Lenti-PD was initiated in subjects (aged 48-70) with Bilateral Idiopathic Parkinson's Disease (PD). Diagnosing was done using the UK Parkinson's Disease Society (PDS) Brain Bank Criteria, but without the specific exclusion of subjects with more than one affected relative. Part A of clinical trial study design is the dose escalation portion as shown in FIG. 4A. Part B of the clinical trial study is the expansion cohort v. imitation surgical procedure portion as shown in FIG. 4B.

Lenti-PD delivers genes encoding three enzymes: the truncated form of human tyrosine hydroxylase (TH), human GTP-cyclohydrolase 1 (CH1), and human aromatic L-amino-acid decarboxylase (AADC) via a single lentiviral vector to encode a set of enzymes required for dopamine synthesis.

Two subjects were dosed at 4.2×10⁶ TU Lenti-PD in the Dose Level 1 cohort of the dose escalation portion of the ongoing Phase 2 study, testing the lowest planned dose of Lenti-PD. Three-month data from this “low dose” cohort (Dose Level 1) was collected. The cohort included two subjects with advanced Parkinson's disease who received a one-time administration of Lenti-PD at the low dose.

Lenti-PD was formulated as a liquid product for striatal infusion with the following: mannitol (1.0% w/v), tromethamine (TRIS) (20 mM), sucrose (1.0%), sodium chloride (100 mM), hydrochloric acid (qs to pH 7.3), and water for injection.

The Lenti-PD was delivered to the subject by local bilateral stereotactic infusion into the sensorimotor putamen of PD subjects allowing for expression of the encoded proteins in non-dopaminergic neurons. Lenti-PD is administered in small, (100 μL) volumes at a controlled rate (about 3 μL/minute) directly into the putamen made via three tracts in the first hemisphere and three tracts in the second hemisphere.

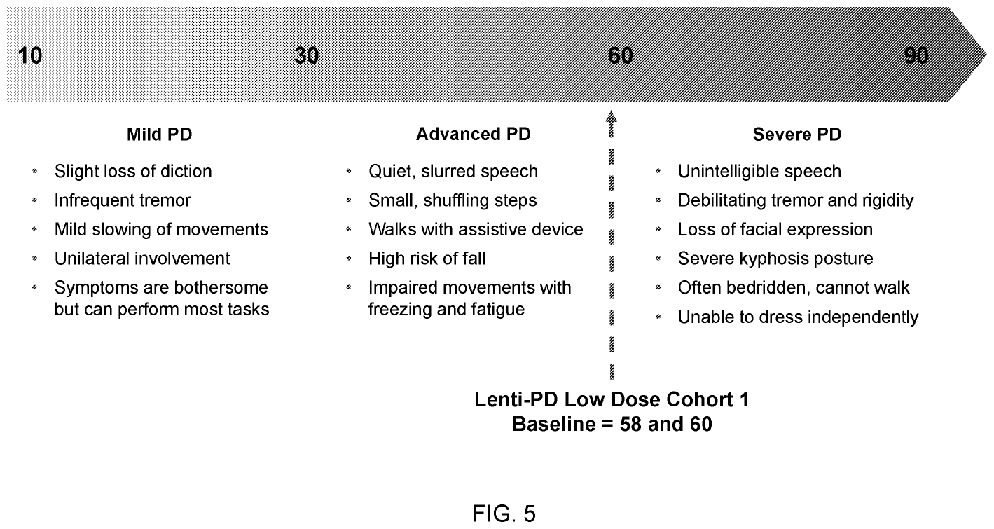

The Unified Parkinson's Disease Rating Scale (UPDRS) score is a physician-rated scale, ranging from 0 to 199 with lower scores indicating improvement. UPDRS Part III (motor) OFF score is an objective, well-validated measure of motor function in Parkinson's disease. The OFF score was assessed while subjects were washed out of oral levodopa therapy. By assessing subjects while they were in the levodopa “off” state, the UPDRS OFF score was designed to capture the benefit of therapy without the potentially confounding effect of background medical treatment. The progression reflected through UPDRS part III (motor) OFF score is shown in FIG. 5. The Dose Level 1 subjects had a baseline UPDRS Part III (motor) OFF score of between 30-60.

Subjects were withdrawn of PD Medication for “OFF”/“ON” Clinical and PET Assessments. The last L-DOPA dose must be at least 12 hours before the “OFF” assessment. For the “ON” assessments, subjects were given a challenge dose of L-DOPA sufficient to achieve an effective “ON” response, which will usually be between 100% and 150% of their usual morning dose. The “ON” assessments was recorded once a good “ON” response had been reached, as judged by subject and examiner. Timing was at least an hour after administration. If the subject's requirement for L-DOPA therapy decreased during the study, the investigator adjusted the L-DOPA challenge dose accordingly.

At three months, subjects in the Dose Level 1 cohort experienced an average UPDRS OFF total score improvement of 54.5 points after receiving Lenti-PD, representing a 55% improvement from baseline.

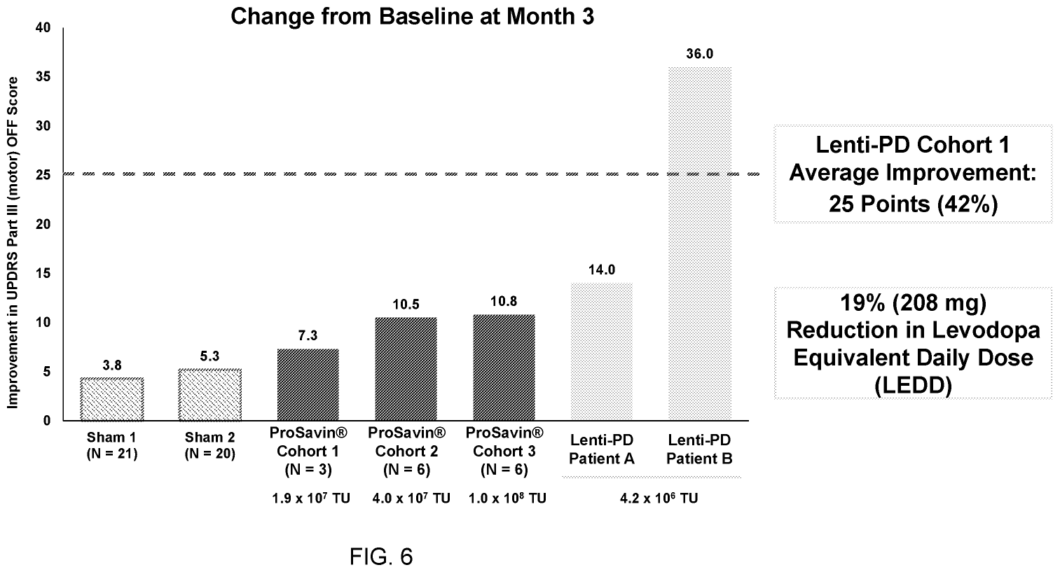

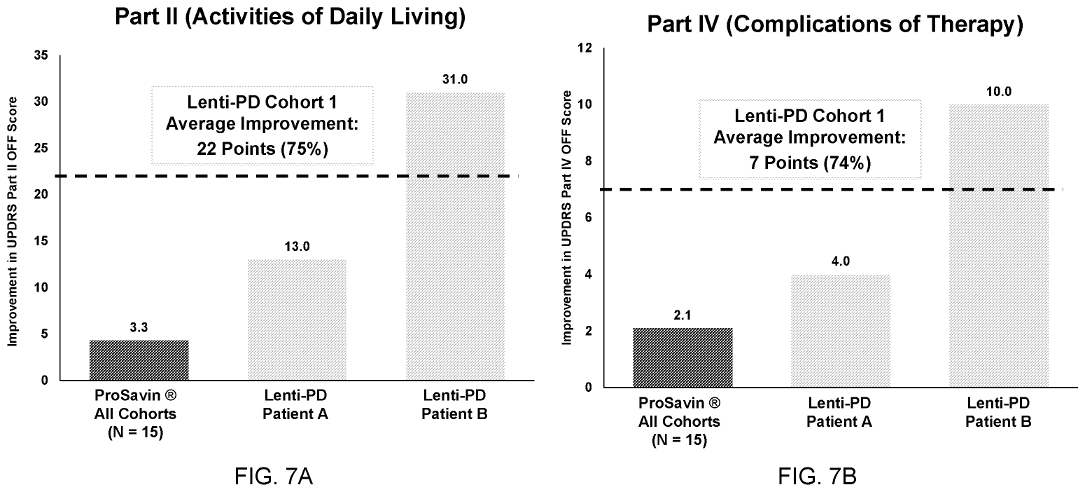

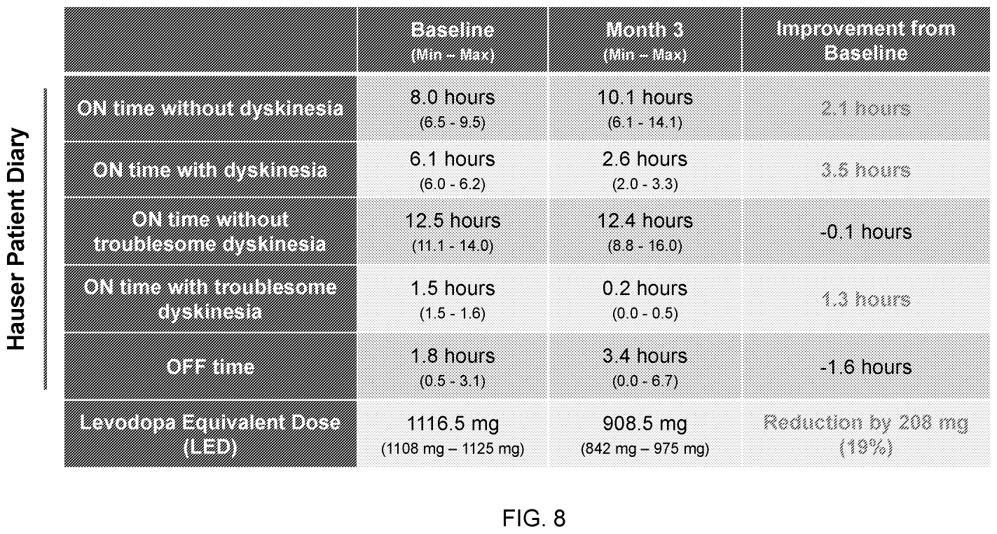

Consistent improvements across multiple UPDRS subscales were also observed. Subjects experienced an improvement of 25 points from baseline on the motor examination subscale (UPDRS Part III OFF) as shown in FIG. 6, an improvement of 22 points from baseline on the activities of daily living subscale (UPDRS Part II OFF) as shown in FIG. 7A, and an improvement of 7 points from baseline on the complications of therapy subscale (UPDRS Part IV OFF) as shown in FIG. 7B.

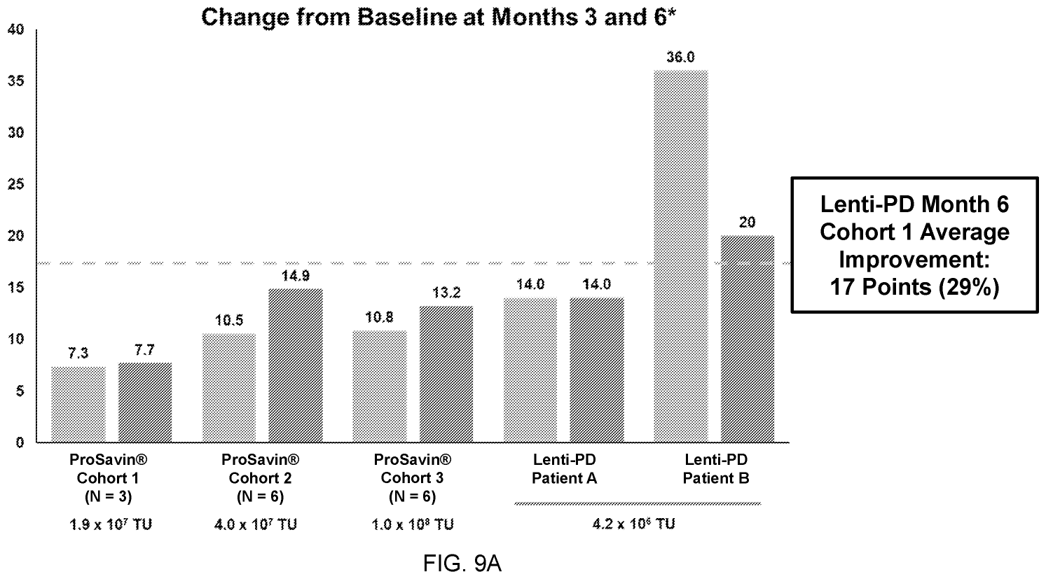

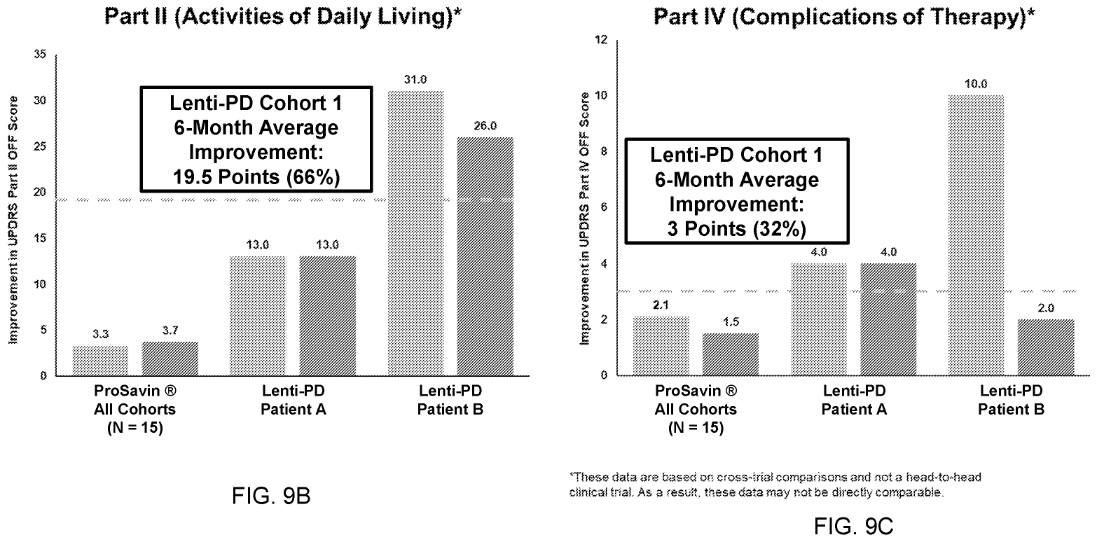

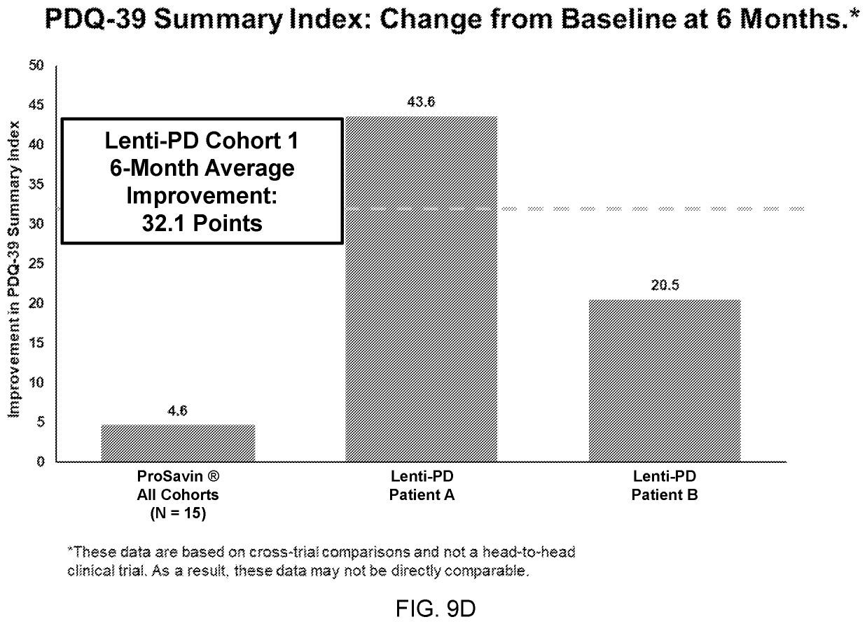

At six months, subjects experienced an improvement of 17 points from baseline on the motor examination subscale (UPDRS Part III OFF) as shown in FIG. 9A, an improvement of 19.5 points from baseline on the activities of daily living subscale (UPDRS Part II OFF) as shown in FIG. 9B, and an improvement of 3 points from baseline on the complications of therapy subscale (UPDRS Part IV OFF) as shown in FIG. 9C. Additionally, a PDQ-39 survey was conducted to assess the change from baseline to three months and six months. PDQ-39 is a questionnaire that assesses Parkinson's disease-specific health related quality. Subjects experiences a 6-month average improvement of 32.1 points as compared to ProSavin® as shown in FIG. 9D. This represents an average total score improvement from baseline of approximately 65%, up from an approximate 37% improvement from baseline as measured at the three-month time point. These data are based on cross-trial comparisons, not a head-to-head clinical trial.

Both Lenti-PD and ProSavin® encode the same three enzymes in the dopamine biosynthetic pathway. A separate completed phase I/II study with ProSavin® was an open label dose escalation study (PS1/001/07, EudraCT 2007-001109-26), and subjects from the study were invited to enroll into a long-term follow on study, (PS1/001/09, EudraCT 2009-017253-35), designed to monitor the safety and tolerability of ProSavin® for up to 10 years post treatment.

Both subjects treated with Lenti-PD in the lowest dose cohort (total dose of 4.2×10⁶ TU) exhibited greater individual improvement in the UPDRS Part III OFF score than the mean improvement observed in any dose cohort of ProSavin® previously tested. Taken together, these results suggest greater efficacy for Lenti-PD at 3 months compared to the highest dose (total dose of 1.0×10⁸ TU) of ProSavin® previously tested. A detailed summary of results on the UPDRS scale for Lenti-PD from this study and for ProSavin® from a prior clinical trial is shown in Table 1 below.

TABLE 1

| | | |
|---|---|---|
| | Lenti-PD Cohort 1 | ProSavin ® Cohort 3 |
| | (Low Dose: 4.2 × 10⁶ TU) | (High Dose: 1.0 × 10⁸ TU) |
| Measure | at 3 months (N = 2) | at 3 months (N = 6) |

UPDRS Total OFF Score. Range from 0 to 199

| | | |
|---|---|---|
| Baseline | 99.0 | 79.3 |
| Month 3 | 44.5 | 61.3 |
| Improvement | 54.5 | 18.0 |
| from Baseline | | |

UPDRS Part I OFF Score (Mentation,
Behavior and Mood). Range from 0 to 16

| | | |
|---|---|---|
| Baseline | 1.0 | 2.5 |
| Month 3 | 0.5 | 0.8 |
| Improvement | 0.5 | 1.7 |
| from Baseline | | |

UPDRS Part II OFF Score (Activities
of Daily Living). Range from 0 to 52

| | | |
|---|---|---|
| Baseline | 29.5 | 21.7 |
| Month 3 | 7.5 | 19.3 |
| Improvement | 22.0 | 2.3 |
| from Baseline | | |

UPDRS Part III OFF Score
(Motor). Range from 0 to 108

| | | |
|---|---|---|
| Baseline | 59.0 | 44.8 |
| Month 3 | 34.0 | 34.0 |
| Improvement | 25.0 | 10.8 |
| from Baseline | | |

UPDRS Part IV OFF Score (Complications
of Therapy). Range from 0 to 23

| | | |
|---|---|---|
| Baseline | 9.5 | 10.3 |
| Month 3 | 2.5 | 7.2 |
| Improvement | 7.0 | 3.2 |
| from Baseline | | |

Subjects continued their standard clinical care for managing their PD medication, including levodopa. At month 3, the average levodopa equivalent dose (LED) reduced by 208 mg for subjects in Dose Level 1. This represents an average reduction of 19% from baseline.

Subjects in Dose Level 1 also experienced an 18% improvement in dyskinesia as measured by the Rush Dyskinesia Rating Scale ON score, an objective assessment of functional disability during activities of daily living while subjects are on oral levodopa. The UPDRS Part IV score, which captures the disability and duration of dyskinesias among other complications of therapy, was also improved as shown in Table 1. Further, the results support lowest tested dose of Lenti-PD had substantially greater biological activity than the highest dose of ProSavin® previously tested.

A subjective subject diary (Hauser Patient Diary) was collected in subjects from Dose Level 1. Although variability was present in the subject-recorded diary entries, both subjects exhibited improvement in diary ON time with dyskinesia, with a mean reduction of 3.5 hours (57%) from baseline. A consistent benefit was also observed in diary ON time with troublesome dyskinesia, with a mean reduction of 1.3 hours (87%) from baseline. Results on the subjective subject diary and Levodopa Equivalent Dose (LED) are summarized in FIG. 8.

The maintenance of a constant dopaminergic tonic level within the putamen offers the potential to reduce the level of L-DOPA and dopamine agonist therapy and provide a sustained motor correction with longer “‘ON” periods and shorter “OFF” periods.

These results suggest that Lenti-PD provide both improvement in motor function (e.g., 25 point (42%) increase in UPDRS-III (motor) OFF benefit achieved at three months, and 17 point (29%) increase in UPDRS-III (motor) OFF benefit achieved at six months) and reduction in dyskinesia (e.g., 87% reduction in diary ON time with troublesome dyskinesia; 74% reduction in UPDRS-IV at three months, 32% reduction in UPDRS-IV at 6 months, and 18% improvement in Rush Dyskinesia) in subjects with advanced Parkinson's disease.

In addition, the patient reported Hauser Diaries were collected at 6 months. On average the patients experienced an improvement from baseline of ON time without dyskinesia of 2.7 hours, an improvement of 3.9 hours in ON time with dyskinesias, an improvement of ON time without troublesome dyskinesias of 0.3 hours, an improvement of ON time with troublesome dyskinesias of 1.5 hours, and a worsening in OFF time of 0.9 hours. At month six, the average levodopa equivalent daily dose (LEDD) was decreased by 233 mg, which represents an average reduction of 21% from baseline. No serious adverse events were observed related to the procedure or vector administration, and Lenti-PD was generally well-tolerated. The total dose to be tested in the second cohort of subjects is 1.4×10⁷ TU.

### Example 3

### Primate Model for Parkinson's Disease Study

A primate study was conducted to assess the efficacy of Lenti-PD in the MPTP macaque model of PD. A separate GLP study was conducted in primates to assess the safety of Lenti-PD. The efficacy study included sixteen Cynomolgous male macaques, aged 4-5 years at the start of the study. The animals were divided into five experimental groups. At 6 months post vector administration all treated animals showed significant improvements in clinical rating scores and spontaneous locomotor activity compared to controls, with the highest recovery observed in the Lenti-PD high dose (HD) group. The vector preparations used in this study are show in Table 2, and the dosage administrations are shown in Table 3. In each study group, 4 animals were treated with the Lenti-PD full dose or a ⅕^{th} dose, ProSavin®, EIAV-LacZ, or EIAV-Null. Both the EIAV-LacZ and EIAV-null vector groups were used as control groups. The summary of the dosages used for each group are detailed in Table 3.

In the GLP safety study six male and six female healthy cynomolgus macaques, aged 3-5 years and weighing 2.1-3.6 kg were recruited to the study. The animals were divided evenly between the control (TSSM buffer) group and the test Lenti-PD group. The study groups and vector assignment for the GLP toxicology study is shown in Table 4.

TABLE 2
Study groups and vector assignment for MPTP efficacy study

| | | Biological | | | |
|---|---|---|---|---|---|
| EIAV | DNA titre | titre | | | |
| Vector | (TU*/m1) | (TU/ml) | Sterility | Mycoplasma | Endotoxin |
| Lenti-PD | 7.62 × 10⁷ | 7.57 × 10⁷ | Pass | Pass | Pass |
| Lenti-PD | 3.67 × 10⁷ | 4.47 × 10⁷ | Pass | Pass | Pass |
| Lenti-PD | 1.09 × 10⁷ | 7.03 × 10⁷ | Pass | Pass | Pass |
| ProSaving ® | 6.12 × 10⁷ | 5.80 × 10⁷ | Pass | Pass | Pass |
| EIAV- | 1.17 × 10⁸ | N/A | Pass | Pass | Pass |
| CMV-Null | | | | | |

*TU—transducing units;
N/A—Not applicable

TABLE 3
Summary of Experimental Group Dosing

| Animal Group | Vector Study | Total vector |
|---|---|---|
| Number | Group | dose/animal |
| 1 | ProSavin ® | 1.22 × 10⁷ |
| 2 | Lenti-PD (FD) | 1.52 × 10⁷ |
| | | 2.18 × 10⁷ |
| 3 | Lenti-PD (LD) | 3.04 × 10⁶ |
| | 1:5 dilution | 4.36 × 10⁶ |
| 4 | EIAV-CMV-LacZ | 5.64 × 10⁷ |
| 5 | EIAV-CMV-Null | 2.34 × 10⁷ |

TABLE 4
Study groups and vector assignment for GLP safety study

| Treatment group | Group size | Total vector dose/animal |
|---|---|---|
| Lenti-PD | 6 (3 male, 3 female) | 7.0 × 10⁶ |
| TSSM buffer (control) | 6 (3 male, 3 female) | — |

### Clinical Scores

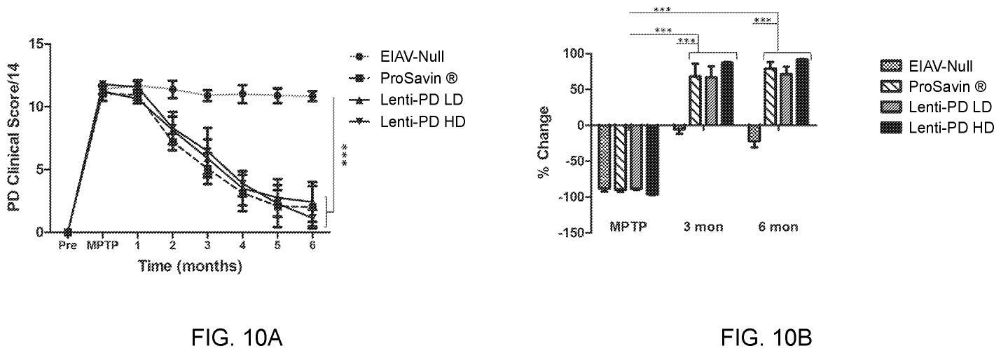

The efficacy study was conducted according to European (EU Directive 86/609/EEC). All efforts were made to minimize animal suffering and animal care was supervised by veterinarians and animal technicians skilled in the healthcare and housing of NHPs. Sixteen adult male cynomolgus monkeys (Macaca fascicularis, supplied by Sicombrec, Philippines) with a mean age of 2.5±0.1 years and a mean weight of 3.48±0.1 kg were housed under standard environmental conditions (12-hour light-dark cycle, temperature: 22±1° C. and humidity: 50%) with free access to food and water. Following MPTP intoxication, all study animals developed Parkinsonian symptoms, with a similar increase in clinical rating scores (CRS) observed across all groups (See FIG. 10A).

Following vector administration, the EIAV-Null group remained stably Parkinsonian, with scores similar to baseline across all time points. For all other treatment groups (ProSavin®, Lenti-PD LD, Lenti-PD HD), progressive improvements in CRS were observed across the 6-month assessment period. All three groups showed significant differences in CRS from the EIAV-Null control group at all time points from 2 to 6 months. A repeated measures ANOVA analysis demonstrated a significant group effect (p=0.0001) and time effect (p<0.0001). A Fisher's LSD post-hoc analysis revealed that all treatment groups were significantly different from the EIAV-Null vector control group (ProSavin®, p=0.0005; Lenti-PD HD, p=0.001; Lenti-PD LD, p=0.001).

### Locomotor Activity

Video-based quantification of locomotor activity was performed on all animals at baseline, post-MPTP intoxication and post vector administration at 3 and 6 months. Following MPTP intoxication, all study animals showed at least a 90%+/− decrease in mean spontaneous locomotor activity, as assessed by total distance moved (TDM) (see FIG. 10B). At 3 and 6 months post vector administration, all three treatment groups showed an increase in TDM relative to post-MPTP values. The Lenti-PD HD groups showed the greatest improvement in TDM at both time-points (87%+/−1 at 3 months and 91%+/−1 at 6 months). A repeated measures ANOVA looking at the differences in locomotor activity as a percentage change from post-MPTP values showed a significant group effect (p=0.0003), time effect (p<0.0001) and time group effect (p<0.0001). A LSD post-hoc analysis revealed that all treatment groups were significantly different from the EIAV-Null vector control group (ProSavin®, p=0.001; Lenti-PD LD, p=0.002; Lenti-PD HD, p<0.001).

### Biodistribution

From each animal in the GLP safety study, samples of buffy coat were collected and analysed at weeks 2 and 4, and plasma samples were collected and analysed at week 2 post treatment by qPCR or qRT-PCR analysis for vector presence. At the end of the study a full macro- and microscopic examination was performed on a wide variety of tissues and organ weights measured. Additional tissue and fluid samples were also collected for vector presence outside the brain and periodic blood sampling was performed throughout the study for Western blot analysis of antibody responses against components of the Lenti-PD vector or transgenes. A full clinical chemistry and urine analysis was also performed on samples obtained pre- and post-vector or buffer administration. Whole body biodistribution analysis showed vector-associated RNA and DNA sequences were not detected in the majority of biological samples from Lenti-PD treated animals. Vector associated DNA sequences were only detected in a small number of samples at a level that was below the lower limit of quantification for the assay. Vector particle (RNA) dissemination or persistence in plasma and shedding in cerebrospinal fluid was absent. There was no indication of a consistent or robust presence of vector associated RNA or DNA sequences.

### Toxicology

The assessment of toxicity against Lenti-PD was based on mortality, clinical signs, body weight, and qualitative food consumption. In addition, in-life assessments were performed by ophthalmoscopy, electrocardiography and blood pressure measurement. A Good Laboratory Practice (GLP) toxicology study investigated the tolerability of bilateral intrastriatal delivery of Lenti-PD vector in normal healthy cynomolgus macaques. The vector used for this study was produced using a Good Manufacturing Practice (GMP) manufacturing process. Over the 26-week observation period Lenti-PD was demonstrated to be well-tolerated and with no clinical signs or abnormal observations noted. Physical examination and assessment of activity, dyskinesia rating, and behavioural scoring revealed no Lenti-PD-related effects. Additionally, there were no treatment-related changes in body weight, appetite, ophthalmoscopy, electrocardiogram (ECG), blood pressure, clinical pathology, macroscopic findings or organ weights. Microscopic findings considered to be related with the treatment of Lenti-PD were minimal to mild perivascular mononuclear cell infiltration with/without pigmented macrophage infiltration at the injection sites. These were not associated with any systemic observations.

All publications mentioned in the above specification are herein incorporated by reference. Various modifications and variations of the described methods and system of the disclosure will be apparent to those skilled in the art without departing from the scope and spirit of the disclosure. Although the disclosure has been described in connection with specific preferred embodiments, it should be understood that the invention as claimed should not be unduly limited to such specific embodiments. Indeed, various modifications of the described modes for carrying out the invention which are obvious to those skilled in molecular biology, virology, neurobiology or related fields are intended to be within the scope of the following claims.

What is claimed is:

1. A method of improving motor function and reducing dyskinesia in a subject suffering from a neurodegenerative disease or a disease where endogenous dopamine levels are reduced in the subject comprising administering an effective amount of a viral vector comprising a nucleic acid construct comprising (i) a nucleotide sequence which encodes tyrosine hydroxylase (TH), (ii) a nucleotide sequence which encodes GTP-cyclohydrolase I (CH1), (iii) a nucleotide sequence which encodes Aromatic Amino Acid Dopa Decarboxylase (AADC), or any combination thereof to the subject.

2. The method of claim 1, wherein the neurodegenerative disease or the disease where endogenous dopamine levels are reduced is Parkinson's Disease.

3. A method of treating or improving motor function and reducing dyskinesia in a subject suffering from Parkinson's Disease comprising administering to the subject a therapeutically effective amount of a composition which comprises: (a) a viral vector comprising a nucleic acid construct comprising (i) a nucleotide sequence which encodes tyrosine hydroxylase (TH), (ii) a nucleotide sequence which encodes GTP-cyclohydrolase I (CH1), (iii) a nucleotide sequence which encodes Aromatic Amino Acid Dopa Decarboxylase (AADC), or any combination thereof; and (b) a pharmaceutically acceptable excipient.

4. The method of claim 2 or 3, wherein the subject is undergoing L-DOPA or LED therapy.

5. The method of any one of claims 1-4, wherein the subject's L-DOPA or LED therapy dose is reduced within three months after administration of the nucleic acid construct relative to the subject's L-DOPA or LED therapy dose prior to administration.

6. The method of any one of claims 4-5, wherein the average L-DOPA or LED therapy dose is reduced by at least 10%, at least 12%, at least 14%, at least 15%, at least 16%, at least 17%, at least 18%, or at least 19% from baseline three months after administration.

7. The method of any one of claims 1-6, wherein the viral vector is administered at a target dose of 1×10⁶ TU/subject to 5×10⁸ TU/subject.

8. The method of claim 7, wherein the target dose is about 4×10⁶ TU/subject to 8×10⁶ TU/subject; 8×10⁶ TU/subject to 4×10⁷ TU/subject; or 1×10⁷ TU/subject to 5×10⁸ TU/subject.

9. The method of any one of claims 1-8, wherein administration is a one-time administration.

10. The method of any one of claims 1-9, wherein administration is to the brain.

11. The method of claim 10, wherein the administration is to the putamen by infusion.

12. The method of any one of claims 1-11 comprising: (i) a nucleotide sequence which encodes tyrosine hydroxylase (TH), (ii) a nucleotide sequence which encodes GTP-cyclohydrolase I (CH1) and (iii) a nucleotide sequence which encodes Aromatic Amino Acid Dopa Decarboxylase (AADC) wherein the nucleotide sequence encoding TH is linked to the nucleotide sequence encoding CH1 such that they encode a fusion protein TH-CH1.

13. The method of any one of claims 1-12, wherein the nucleic acid construct comprises:
TH-_{L}-CH1-_{IRES}-AADC;
AADC-_{L}-TH-_{L}-CH1;
TH-_{L}-CH1-_{L}-AADC;
or TH-_{L}-CH1-_{L}-AADC;
wherein L is a linker-encoding sequence, IRES is an Internal Ribosome Entry Site, and P is a promoter.

14. The method of claim 13, wherein the nucleic acid construct comprises TH-_{L}-CH1-_{IRES}-AADC.

15. The method of any one of claims 1-14, wherein the nucleic acid construct comprises (i) a nucleotide sequence which encodes tyrosine hydroxylase (TH), (ii) a nucleotide sequence which encodes GTP-cyclohydrolase I (CH1) and (iii) a nucleotide sequence which encodes Aromatic Amino Acid Dopa Decarboxylase (AADC), wherein the nucleotide sequence encoding TH is linked to the nucleotide sequence encoding CH1 such that they encode a fusion protein TH-CH1, wherein the construct comprises TH-L-CH1-IRES-AADC or TH-L-CH1-P-AADC, wherein L is a linker-encoding sequence, IRES is an Internal Ribosome Entry Site, and P is a promoter.

16. The method of any one of claims 13-15, wherein the linker (L) is not codon optimized.

17. The method of any one of claims 13-16, wherein the linker (L) comprises the sequence shown as SEQ ID NO: 1 or SEQ ID NO: 3.

18. The method of any one of claims 1-17, wherein the nucleic acid construct further comprises a promoter (P) selected from a constitutive promoter, a tissue-specific promoter, or a combination thereof.

19. The method of claim 18, wherein the promoter is a CMV promoter, a phosphoglycerate kinase promoter or a thymidine kinase promoter.

20. The method of any one of claims 1-19, wherein the viral vector is a lentiviral vector or an adeno-associated viral vector.

21. The method of claim 20, wherein the viral vector is a lentiviral vector.

22. The method of any one of claims 1-21, wherein the viral vector is a viral particle.

23. The method of claim 22, wherein the viral particle is an EIAV vector particle, and which is pseudotyped with VSV-G.

24. The method of any one of claims 1-23, wherein the viral vector is formulated as pharmaceutical composition comprising a pharmaceutically acceptable excipient and/or diluent.

25. The method of any one of claims 1-24, wherein motor function in the subject is improved as shown by at least a 25%, at least a 30%, at least a 35%, or at least a 40% increase in UPDRS-III (motor) OFF score 3 months after administration compared to baseline.

26. The method of any one of claims 1-25, wherein dyskinesias is reduced in the subject as shown by one or more of the following: (a) at least 50%, at least 60%, at least 70%, at least 75%, at least 80%, or at least 85% reduction in Hauser diary ON time with troublesome dyskinesia 3 months after administration compared to baseline; (b) at least 50% reduction, at least 60%, at least 65%, at least 70%, or at least 74% reduction in UPDRS-IV score 3 months after administration compared to baseline, and (c) at least 10%, at least 12%, at least 15%, at least 18% improvement in Rush Dyskinesia score 3 months after administration compared to baseline.

27. The method of any one of claims 1-26, wherein the method further improves one or more symptoms in the subject selected from the group consisting of tremors, bradykinesia, rigid muscles, impaired posture and/or balance, loss of automatic movements, difficulty speaking, difficulty with fine motor skills, or any combination thereof.

28. The method of any one of claims 1-27, wherein within three months after administration, the subject has improved motor function, reduced dyskinesia, and reduced L-DOPA or LED therapy dose after administration relative to the subject's baseline before administration.


SEQUENCE LISTING (10 sequences)

SEQ ID NO: 1 - DNA, 45 bp, Artificial Sequence
  Synthetic linker-encoding oligonucleotide
ggaggtggcgggtccgggggcgggggtagcggtggcgggggctcc

SEQ ID NO: 2 - protein, 15 aa, Artificial Sequence
  Synthetic linker peptide
GGGGSGGGGSGGGGS

SEQ ID NO: 3 - DNA, 45 bp, Artificial Sequence
  Synthetic linker-encoding oligonucleotide
gggggaggcggtagcggcggagggggctccggcggaggcgggagc

SEQ ID NO: 4 - protein, 25 aa, Artificial Sequence
  Synthetic linker peptide
GGGGSGGGGSGGGGSGGGGSGGGGS

SEQ ID NO: 5 - protein, 21 aa, Artificial Sequence
  Synthetic linker peptide
NFIRGREDLLEKIIRQKGSSN

SEQ ID NO: 6 - protein, 23 aa, Artificial Sequence
  Synthetic linker peptide
NLSSDSSLSSPSALNSPGIEGLS

SEQ ID NO: 7 - protein, 14 aa, Artificial Sequence
  Synthetic linker peptide
QGATFALRGDNPQG

SEQ ID NO: 8 - protein, 18 aa, Artificial Sequence
  Synthetic linker peptide
SGGGEILDVPSTGGSSPG

SEQ ID NO: 9 - DNA, 148 bp, Artificial Sequence
  Synthetic polynucleotide
  misc_feature (1)..(148): Truncated TH linked to AADC via a GS15 linker
ggcgctccctggagggtgtccaggatgagctggacacccttgcccatgcgctgagcgccatcggcgggggaggcggtagcggcggagggggctccggcggaggcgggagcatggacgccagtgagttccgaaggcgcggcaaggagat

SEQ ID NO: 10 - protein, 50 aa, Artificial Sequence
  Synthetic polypeptide
  MISC_FEATURE (1)..(50): Truncated TH linked to AADC via a GS15 linker
RRSLEGVQDELDTLAHALSAIGGGGGSGGGGSGGGGSMDASEFRRRGKEM